(19) United States
(12) Patent Application Publication
(10) Pub. No.: US 2004/0132158 A1
(43) Pub. Date: Jul. 8, 2004

(54) Human peptidases

(75) Inventors: Olga Bandman, Mountain View, CA (US); Jennifer L. Hillman, Santa Cruz, CA (US); Y. Tom Tang, San Jose, CA (US); Preeti Lal, Santa Clara, CA (US); Henry Yue, Sunnyvale, CA (US); Yalda Azimzai, Oakland, CA (US); Mariah R. Baughn, Los Angeles, CA (US); Dyung Aina M. Lu, San Jose, CA (US)

Correspondence Address: INCYTE CORPORATION, 3160 PORTER DRIVE, PALO ALTO, CA, 94304, US

(73) Assignee: Incyte Corporation, Palo Alto, CA

(21) Appl. No.: 10/729,807

(22) Filed: Dec. 5, 2003

Related U.S. Application Data
(62) Division of application No. 09/889,238, filed on Jan. 24, 2002, now abandoned.
Application No. 09/889,238 is a 371 of international application No. PCT/US00/00641, filed on Jan. 11, 2000.
(60) Provisional application No. 60/172,247, filed on Jan. 11, 1999.
(60) Provisional application No. 60/132,253, filed on May 3, 1999.
(60) Provisional application No. 60/136,653, filed on May 27, 1999.

(51) Int. Cl. C12N 9/64; C07H 21/04

(52) U.S. Cl. 435/226; 435/69.1; 435/320.1; 435/325; 536/23.2

(57) ABSTRACT

The invention provides human peptidases (HPEP) and polynucleotides which identify and encode HPEP. The invention also provides expression vectors, host cells, antibodies, agonists, and antagonists. The invention also provides methods for diagnosing, treating, or preventing disorders associated with expression of HPEP.

This application is a divisional application of U.S. application Ser. No. 09/889,238, filed Jan. 24, 2002, entitled HUMAN PEPTIDASES, which is a 371 of PCT/US00/00641, filed Jan. 11, 2000, which claims domestic priority to U.S. application Ser. No. 60/172,247, filed Jan. 11, 1999, entitled HUMAN PEPTIDASES; U.S. application Ser. No. 60/132,253, filed May 3, 1999, entitled PROTEIN DEGRADATION MOLECULES; and U.S. application Ser. No. 60/136,653, filed May 27, 1999, entitled HUMAN ENDOPEPTIDASE MOLECULES, the contents of all of which are hereby expressly incorporated by reference herein.

### TECHNICAL FIELD

This invention relates to nucleic acid and amino acid sequences of human peptidases and to the use of these sequences in the diagnosis, treatment, and prevention of cell proliferative, autoimmune/inflammatory, and metabolic disorders.

### BACKGROUND OF THE INVENTION

Peptidases, also called proteases, are enzymes which cleave the peptide bonds forming the backbones of peptides and proteins. Peptidases are required to control the turnover of cellular proteins, which typically have half-lives ranging from hours to a few days. The cleavage of peptide bonds within cells is necessary for the maturation of precursor proteins to their active forms, the removal of signal sequences from targeted proteins, and the degradation of incorrectly folded proteins. Regulated proteolysis and protein degradation by peptidases are essential for normal cell growth, embryonic development, differentiation, wound healing, tissue remodeling, apoptosis, and homeostasis, as well as inflammation and immune response. Peptidases are necessary components of bacterial, parasitic, and viral invasion and replication within a host. Mammalian peptidases have been identified and categorized based on active site structure, mechanism of action, and three-dimensional structure. (See, e.g., Beynon, R. J. and J. S. Bond (1994) Proteolytic Enzymes: A Practical Approach, Oxford University Press, New York N.Y., pp. 1-5.)

The serine proteases (SPs) are a large family of peptidases that include the digestive enzymes trypsin and chymotrypsin; components of the complement and blood-clotting cascades; and enzymes that control the degradation and turnover of macromolecules of the extracellular matrix. SPs are so named because of the presence of a serine residue, usually within a conserved sequence, in the catalytic active site. This catalytic serine forms a triad together with an aspartate and a histidine residue. The main SP sub-families are trypases, which cleave peptide backbones after an arginine or a lysine residue; aspartases, which cleave after aspartate; chymases, which cleave after phenylalanine or leucine; metases, which cleavage after methionine; and serases, which cleave after serine. Pancreatic serine proteases are secreted from the pancreas into the duodenum where they degrade proteins ingested in food. Examples of these proteases include chymotrypsin, trypsin, elastase, and pancreatic kallikrein. Prolylcarboxypeptidase, a lysosomal SP that cleaves peptides such as angiotensin II and III and [des-Arg9] bradykinin, shares sequence homology with members of both the serine carboxypeptidase and prolylendopeptidase families (Tan, F. et al. (1993) J. Biol. Chem. 268:16631-16638). Plasma serine proteases, which include thrombin and Clr, are involved in blood coagulation and immune response. Thrombin converts fibrinogen, a large soluble plasma protein, into fibrin, a smaller insoluble protein that aggregates to form blood clots. C1r is a component of the complement system, a complex of proteins that perforates the cell membranes of invading microorganisms.

Defects in SPs or their associated regulatory factors are involved in a range of human diseases, including hemorrhagic disorders, thrombophilia, immune disorders, and pancreatic deficiency. For example, mutations in a serine protease cofactor, factor VIII, are the cause of hemophilia. In contrast, excessive expression of the SP prothrombin is one cause of thrombophilia, a genetic predisposition to develop blood clots (Kato, G. J. (1999) Hum. Mutat. 13:87-98). Most mammalian serine proteases are synthesized as zymogens, inactive precursors that are activated by protease cascades. For example, trypsinogen is converted to its active form, trypsin, by enterokinase. Enterokinase, the initiator of intestinal digestion, is an SP found in the intestinal brush border, where it removes an N-terminal fragment from trypsinogen to yield active trypsin (Kitamoto, Y. et al. (1994) Proc. Natl. Acad. Sci. USA 91:7588-7592). In turn, trypsin activates the precursors of the other pancreatic enzymes. Mutations in enterokinase result in severe pancreatic exocrine deficiency (Kato, supra).

The cysteine proteases (CPs) are peptidases involved in diverse cellular processes ranging from the processing of precursor proteins to intracellular degradation. CPs have a cysteine as the major catalytic residue in an active site where catalysis proceeds via a thiol ester intermediate and is facilitated by adjacent histidine and aspartic acid residues. Mammalian CPs include lysosomal cathepsins and cytosolic calcium activated proteases (calpains). Cysteine proteases are produced by monocytes, macrophages and other cells of the immune system which migrate to sites of inflammation and, in their protective role, secrete various molecules to repair damaged tissue. Without proper regulation, these cells may overproduce the same molecules and cause tissue destruction in certain disorders. In autoimmune diseases such as rheumatoid arthritis, the secretion of the cysteine protease cathepsin C degrades collagen, laminin, elastin and other structural proteins found in the extracellular matrix of bones. The cathepsin family of lysosomal proteases includes cysteine proteases (cathepsins B, H, K, L, O2, and S) and aspartyl proteases (cathepsins D and E). Various members of this endosomal peptidase family are differentially expressed. Some, such as cathepsin D, have a ubiquitous tissue distribution while others, such as cathepsin L, are found only in monocytes, macrophages, and other cells of the immune system.

Aspartic proteases (APs) are distinguished from the SPs and CPs by the presence of a pair of aspartic acid residues in the active site, and are most active in the pH 2-3 range, in which one of the aspartate residues is ionized, and the other aspartate is not ionized. APs include penicillopepsin, mammalian pepsin, pepsin A, gastricsin, chymosin, renin, certain fungal peptidases, and members of the cathepsin family of lysosomal proteases such as cathepsins D and E.

Metalloproteases are peptidases which use zinc as an active site component. The zinc atoms of metalloproteases are bound into the enzyme active site by two glutamic acid residues and one histidine residue. Metalloproteases are most notably represented in mammals by the exoproteases carboxypeptidase A and B, and the matrix metalloproteases (MMPs). Carboxypeptidases A and B have similar structures and active sites. Carboxypeptidase A, like chymotrypsin, prefers C-terminal aromatic and aliphatic side chains of hydrophobic nature, whereas carboxypeptidase B is directed toward basic arginine and lysine residues. Another metalloprotease is glycoprotease (GCP), or O-sialoglycoprotein endopeptidase, a peptidase which specifically cleaves O-sialoglycoproteins such as glycophorin A. Placental leucine aminopeptidase (P-LAP) is a metalloprotease which degrades several peptide hormones such as oxytocin and vasopressin, suggesting a role in maintaining homeostasis during pregnancy, and is expressed in several tissues, some of which express two forms of P-LAP mRNAs (Rogi, T. et al. (1996) J. Biol. Chem. 271:56-61).

MMPs are a family of endopeptidases that play an important role in remodeling of the extracellular matrix (ECM). This family includes the collagenases, gelatinases, and stromelysins. MMPs are involved in both normal and pathological tissue remodeling processes including wound healing, inflammation, post-lactational mammary gland involution, and trophoblast invasion during implantation. (See, e.g., Shapiro, S. D. (1998) Curr. Opin. Cell Biol. 10:602-608; Birkedal-Hansen, H. (1995) Curr. Opin. Cell Biol. 7:728-735.) MMPs contribute to the progression of various diseases including arthritis, atherosclerosis, and cancer. MMPs are key players in the irreversible degradation of the ECM seen in rheumatic disease. In cells isolated from inflamed synovia, the mRNAs for stromelysin, cytokines, TIMP-1, cathepsin, gelatinase, and other molecules are preferentially expressed (Keyszer, G. M. (1995) Arthritis Rheum. 38:976-984). A genetic polymorphism which causes diminished expression of stromelysin-1 is associated with enhanced progression of atherosclerosis, a chronic inflammatory process in which plaques are formed in the arterial vessel walls by the accumulation of ECM, smooth muscle cells, and lipid-laden macrophages (Ye, S. et al. (1996) J. Biol. Chem. 271:13055-13060). MMPs play a critical role in tumor invasion and metastasis, helping the tumor to spread by breaking down the surrounding ECM. Overexpression of MMP-3 in mice leads to an increased incidence of breast cancers, while deletions of MMPs suppress tumorigenesis (Sympson, C. J. et al. (1995) Semin. Cancer Biol. 6:159-163; Shapiro, supra). Synthetic MMP inhibitors are currently being tested in clinical trials against breast cancer (Brown, P. D. (1998) Breast Cancer Res. Treat. 52:125-136).

MMPs are regulated in cells by the tissue inhibitors of metalloproteinases (TIMPs). Mutations in TIMP-3 in humans lead to Sorsby's fundus dystrophy, a hereditary degenerative disease of the retina (Weber, B. H. et al. (1994) Nat. Genet. 8:352-356). TIMPs are involved in inhibition of tumor invasion, as overexpression of TIMPs can decrease tumor progression in animal models, and TIMPs also play a role in regulation of cell growth (Shapiro, supra; Birkedal-Hansen, supra). Overexpresssion of TIMP-3 inhibits tumor invasion in vitro and promotes cell death of different cancer cell types, making it potentially useful for gene therapy of multiple cancer types (Baker, A. H. et al. (1999) Br. J. Cancer 79:1347-1355).

Characteristic sequence motifs in addition to the conserved active site motifs are observed in peptidases. Some SPs contain Kringle domains, triple-looped disulfide cross-linked domains that may function in binding membranes, other proteins or phospholipids, or in the regulation of proteolytic activity. Two plasma serine proteases, plasma kallikrein and coagulation factor XI, have a C-terminal catalytic domain and four tandem N-terminal repeats of about 90 amino acids, including 6 conserved cysteines. Three disulfide bonds linking the first and sixth, second and fifth, and third and fourth cysteines to produce a globular “apple domain.”

As an alternative to structure-based classification, peptidases may also be classified by function. Functional classes include the aminopeptidases and signal peptidases. Aminopeptidases catalyze the hydrolysis of amino acid residues from the amino terminus of peptide substrates. Bovine leucine aminopeptidase is a zinc metalloprotease that utilizes the sulfydryl groups from at least three reactive cysteine residues at its active site in the binding of metal ions (Cuypers, H. T. et al. (1982) J. Biol. Chem. 257:7086-7091). Signal peptidases are a specialized class of peptidases that serve in the processing of signal peptides, the amino-terminal sequences which direct a protein from its ribosomal assembly site to a particular cellular or extracellular location. After export, a signal peptidase removes the signal sequence. Signal peptidases exist as multi-subunit complexes in both yeast and mammals.

The ubiquitin-proteasome pathway regulates the proteolysis of cell cycle and growth regulators, including mitotic cyclic kinases; components of signal transduction pathways, including cell surface receptors; transcriptional regulators; oncoproteins; tumor suppressor genes such as p53; viral proteins; and mutated or damaged proteins (Ciechanover, A. (1994) Cell 79:13-21). The system also processes antigens for presentation by the major histocompatability complex class I molecules. Proteins are targeted for degradation by the covalent attachment of multiple molecules of ubiquitin, a small, heat-stable protein, to a lysine residue on the target protein. Attachment of ubiquitin to target proteins is mediated by a member of the ubiquitin ligase family. The ubiquitin-tagged proteins are then recognized and degraded by the proteasome, a large (˜2000 kDa), multisubunit complex composed of a central catalytic core containing a variety of peptidases and terminal subunits that serve in substrate recognition and regulation of proteasome activity. During this process, ubiquitin is released from the target proteins and reutilized.

Proteins involved in the ubiquitin-proteasome pathway have been implicated in specific diseases. Certain cell cycle regulators are recognized by multisubunit ubiquitin ligase complexes that include F-box domain proteins which mediate the recruitment of specific substrates for ubiquitination. Mutations in the ubiquitin ligase enzyme E6-AP are the cause of Angelman's syndrome, a neurological disorder characterized by mental retardation, seizures, and poor coordination and muscle tone. E6-AP is also the target of E6, a viral protein, produced by strains of the human papilloma virus, associated with cervical cancer. E6 modifies the function of E6-AP to accelerate the degradation of the tumor suppressor protein p53 (Ciechanover, A. (1998) EMBO J. 17:7151-7160; Kato, G. J. (1999) Hum. Mutat. 13:87-98). A murine proto-oncogene, Unp, encodes a nuclear ubiquitin protease whose overexpression leads to oncogenic transformation of NIH3T3 cells, and the human homolog of this gene is consistently elevated in small cell tumors and adenocarcinomas of the lung (Gray, D. A. (1995) Oncogene 10:2179-2183).

Protease inhibitors play a major role in the regulation of the activity and effect of peptidases. For example, the secretory leukocyte protease inhibitor (SLPI) is secreted by epithelial cells and neutrophils, and inhibits leukocyte-secreted serine proteases including elastase and cathepsin G from neutrophils, chymase and trypsin from mast cells, and trypsin and chymotrypsin from pancreatic acinar cells. SLPI and related protease inhibitors are characterized by a four disulfide core structure, or whey acidic protein (WAP) domain. SLPI suppresses the macrophage response to bacterial lipopolysaccharide, which can cause tissue injury, circulatory failure, multiple organ failure, and death. Together with α-1 protease inhibitor, SLPI protects the lungs from emphysema induced by neutrophil elastase. SLPI also possesses antimicrobial activity against fungi, bacteria and HIV (Jin, F. -Y. et al. (1997) Cell 88:417-426; Tomee, J. F. et al. (1998) Thorax 53:114-116).

Cystatins, inhibitors of cysteine proteases, have been associated with a variety of disorders. Low levels of cystatins seem to be correlated with malignant progression of tumors (Calkins, C. et al. (1998) J. Histochem. Cytochem. 46:745-751; Hoppe-Seyler, F. and K. J. Butz (1995) J. Mol. Med. 73:529-538). Increased cysteine protease levels, when accompanied by reductions in inhibitor activity, are correlated with increased malignant properties of tumor cells and the pathology of arthritis and immunological diseases.

The discovery of new human peptidases and the polynucleotides encoding them satisfies a need in the art by providing new compositions which are useful in the diagnosis, prevention, and treatment of cell proliferative, autoimmune/inflammatory, and metabolic disorders.

### SUMMARY OF THE INVENTION

The invention features purified polypeptides, human peptidases, referred to collectively as “HPEP” and individually as “HPEP-1,” “HPEP-2,” “HPEP-3,” “HPEP-4,” “HPEP-5,” “HPEP-6,” “HPEP-7,” “HPEP-8,” “HPEP-9,” “HPEP-10,” “HPEP-11,” “HPEP-12,” “HPEP-13,” “HPEP-14,” “HPEP-15,” “HPEP-16,” “HPEP-17,” and “HPEP-18.” In one aspect, the invention provides an isolated polypeptide comprising a) an amino acid sequence selected from the group consisting of SEQ ID NO:1-18, b) a naturally occurring amino acid sequence having at least 90% sequence identity to an amino acid sequence selected from the group consisting of SEQ ID NO:1-18, c) a biologically active fragment of an amino acid sequence selected from the group consisting of SEQ ID NO:1-18, or d) an immunogenic fragment of an amino acid sequence selected from the group consisting of SEQ ID NO:1-18. In one alternative, the invention provides an isolated polypeptide comprising the amino acid sequence of SEQ ID NO:1-18.

The invention further provides an isolated polynucleotide encoding a polypeptide comprising a) an amino acid sequence selected from the group consisting of SEQ ID NO:1-18, b) a naturally occurring amino acid sequence having at least 90% sequence identity to an amino acid sequence selected from the group consisting of SEQ ID NO:1-18, c) a biologically active fragment of an amino acid sequence selected from the group consisting of SEQ ID NO:1-18, or d) an immunogenic fragment of an amino acid sequence selected from the group consisting of SEQ ID NO:1-18. In one alternative, the polynucleotide is selected from the group consisting of SEQ ID NO:19-36.

Additionally, the invention provides a recombinant polynucleotide comprising a promoter sequence operably linked to a polynucleotide encoding a polypeptide comprising a) an amino acid sequence selected from the group consisting of SEQ ID NO:1-18, b) a naturally occurring amino acid sequence having at least 90% sequence identity to an amino acid sequence selected from the group consisting of SEQ ID NO:1-18, c) a biologically active fragment of an amino acid sequence selected from the group consisting of SEQ ID NO:1-18, or d) an immunogenic fragment of an amino acid sequence selected from the group consisting of SEQ ID NO:1-18. In one alternative, the invention provides a cell transformed with the recombinant polynucleotide. In another alternative, the invention provides a transgenic organism comprising the recombinant polynucleotide.

The invention also provides a method for producing a polypeptide comprising a) an amino acid sequence selected from the group consisting of SEQ ID NO:1-18, b) a naturally occurring amino acid sequence having at least 90% sequence identity to an amino acid sequence selected from the group consisting of SEQ ID NO:1-18, c) a biologically active fragment of an amino acid sequence selected from the group consisting of SEQ ID NO:1-18, or d) an immunogenic fragment of an amino acid sequence selected from the group consisting of SEQ ID NO:1-18. The method comprises a) culturing a cell under conditions suitable for expression of the polypeptide, wherein said cell is transformed with a recombinant polynucleotide comprising a promoter sequence operably linked to a polynucleotide encoding the polypeptide, and b) recovering the polypeptide so expressed.

Additionally, the invention provides an isolated antibody which specifically binds to a polypeptide comprising a) an amino acid sequence selected from the group consisting of SEQ ID NO:1-18, b) a naturally occurring amino acid sequence having at least 90% sequence identity to an amino acid sequence selected from the group consisting of SEQ ID NO:1-18, c) a biologically active fragment of an amino acid sequence selected from the group consisting of SEQ ID NO:1-18, or d) an immunogenic fragment of an amino acid sequence selected from the group consisting of SEQ ID NO:1-18.

The invention further provides an isolated polynucleotide comprising a) a polynucleotide sequence selected from the group consisting of SEQ ID NO:19-36, b) a naturally occurring polynucleotide sequence having at least 90% sequence identity to a polynucleotide sequence selected from the group consisting of SEQ ID NO:19-36, c) a polynucleotide sequence complementary to a), or d) a polynucleotide sequence complementary to b). In one alternative, the polynucleotide comprises at least 60 contiguous nucleotides.

Additionally, the invention provides a method for detecting a target polynucleotide in a sample, said target polynucleotide having a sequence of a polynucleotide comprising a) a polynucleotide sequence selected from the group consisting of SEQ ID NO:19-36, b) a naturally occurring polynucleotide sequence having at least 90% sequence identity to a polynucleotide sequence selected from the group consisting of SEQ ID NO:19-36, c) a polynucleotide sequence complementary to a), or d) a polynucleotide sequence complementary to b). The method comprises a) hybridizing the sample with a probe comprising at least 16 contiguous nucleotides comprising a sequence complementary to said target polynucleotide in the sample, and which probe specifically hybridizes to said target polynucleotide, under conditions whereby a hybridization complex is formed between said probe and said target polynucleotide, and b) detecting the presence or absence of said hybridization complex, and optionally, if present, the amount thereof. In one alternative, the probe comprises at least 30 contiguous nucleotides. In another alternative, the probe comprises at least 60 contiguous nucleotides.

The invention further provides a pharmaceutical composition comprising an effective amount of a polypeptide comprising a) an amino acid sequence selected from the group consisting of SEQ ID NO:1-18, b) a naturally occurring amino acid sequence having at least 90% sequence identity to an amino acid sequence selected from the group consisting of SEQ ID NO:1-18, c) a biologically active fragment of an amino acid sequence selected from the group consisting of SEQ ID NO:1-18, or d) an immunogenic fragment of an amino acid sequence selected from the group consisting of SEQ ID NO:1-18, and a pharmaceutically acceptable excipient. The invention additionally provides a method of treating a disease or condition associated with decreased expression of functional HPEP, comprising administering to a patient in need of such treatment the pharmaceutical composition.

The invention also provides a method for screening a compound for effectiveness as an agonist of a polypeptide comprising a) an amino acid sequence selected from the group consisting of SEQ ID NO:1-18, b) a naturally occurring amino acid sequence having at least 90% sequence identity to an amino acid sequence selected from the group consisting of SEQ ID NO:1-18, c) a biologically active fragment of an amino acid sequence selected from the group consisting of SEQ ID NO:1-18, or d) an immunogenic fragment of an amino acid sequence selected from the group consisting of SEQ ID NO:1-18. The method comprises a) exposing a sample comprising the polypeptide to a compound, and b) detecting agonist activity in the sample. In one alternative, the invention provides a pharmaceutical composition comprising an agonist compound identified by the method and a pharmaceutically acceptable excipient. In another alternative, the invention provides a method of treating a disease or condition associated with decreased expression of functional HPEP, comprising administering to a patient in need of such treatment the pharmaceutical composition.

Additionally, the invention provides a method for screening a compound for effectiveness as an antagonist of a polypeptide comprising a) an amino acid sequence selected from the group consisting of SEQ ID NO:1-18, b) a naturally occurring amino acid sequence having at least 90% sequence identity to an amino acid sequence selected from the group consisting of SEQ ID NO:1-18, c) a biologically active fragment of an amino acid sequence selected from the group consisting of SEQ ID NO:1-18, or d) an immunogenic fragment of an amino acid sequence selected from the group consisting of SEQ ID NO:1-18. The method comprises a) exposing a sample comprising the polypeptide to a compound, and b) detecting antagonist activity in the sample. In one alternative, the invention provides a pharmaceutical composition comprising an antagonist compound identified by the method and a pharmaceutically acceptable excipient. In another alternative, the invention provides a method of treating a disease or condition associated with overexpression of functional HPEP, comprising administering to a patient in need of such treatment the pharmaceutical composition.

The invention further provides a method for screening a compound for effectiveness in altering expression of a target polynucleotide, wherein said target polynucleotide comprises a sequence selected from the group consisting of SEQ ID NO:19-36, the method comprising a) exposing a sample comprising the target polynucleotide to a compound, and b) detecting altered expression of the target polynucleotide.

### BRIEF DESCRIPTION OF THE FIGURES AND TABLES

FIGS. 1A, 1B, 1C, 1D, and 1E show the amino acid sequence alignment between HPEP-1 (Incyte Clone ID 155179; SEQ ID NO:1) and human enterokinase (GI 746413; SEQ ID NO:37), produced using the multisequence alignment program of LASERGENE software (DNASTAR, Madison Wis.).

FIGS. 2A, 2B, and 2C show the amino acid sequence alignment between HPEP-2 (Incyte Clone ID 2415780; SEQ ID NO:2) and Methanococcus jannaschii O-sialoglycoprotein endopeptidase (GI 2826367; SEQ ID NO:38), produced using the multisequence alignment program of LASERGENE software.

FIGS. 3A, 3B, and 3C show the amino acid sequence alignment between HPEP-3 (Incyte Clone ID 2879274; SEQ ID NO:3) and human prolylcarboxypeptidase (GI 431321; SEQ ID NO:39), produced using the multisequence alignment program of LASERGENE software.

Table 1 shows polypeptide and nucleotide sequence identification numbers (SEQ ID NOs), clone identification numbers (clone IDs), cDNA libraries, and cDNA fragments used to assemble full-length sequences encoding HPEP.

Table 2 shows features of each polypeptide sequence, including potential motifs, homologous sequences, and methods, algorithms, and searchable databases used for analysis of HPEP.

Table 3 shows selected fragments of each nucleic acid sequence; the tissue-specific expression patterns of each nucleic acid sequence as determined by northern analysis; diseases, disorders, or conditions associated with these tissues; and the vector into which each cDNA was cloned.

Table 4 describes the tissues used to construct the cDNA libraries from which cDNA clones encoding HPEP were isolated.

Table 5 shows the tools, programs, and algorithms used to analyze HPEP, along with applicable descriptions, references, and threshold parameters.

### DESCRIPTION OF THE INVENTION

Before the present proteins, nucleotide sequences, and methods are described, it is understood that this invention is not limited to the particular machines, materials and methods described, as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a,” “an,” and “the” include plural reference unless the context clearly dictates otherwise. Thus, for example, a reference to “a host cell” includes a plurality of such host cells, and a reference to “an antibody” is a reference to one or more antibodies and equivalents thereof known to those skilled in the art, and so forth.

Unless defined otherwise, all technical and scientific terms used herein have the same meanings as commonly understood by one of ordinary skill in the art to which this invention belongs. Although any machines, materials, and methods similar or equivalent to those described herein can be used to practice or test the present invention, the preferred machines, materials and methods are now described. All publications mentioned herein are cited for the purpose of describing and disclosing the cell lines, protocols, reagents and vectors which are reported in the publications and which might be used in connection with the invention. Nothing herein is to be construed as an admission that the invention is not entitled to antedate such disclosure by virtue of prior invention.

Definitions

“HPEP” refers to the amino acid sequences of substantially purified HPEP obtained from any species, particularly a mammalian species, including bovine, ovine, porcine, murine, equine, and human, and from any source, whether natural, synthetic, semi-synthetic, or recombinant.

The term “agonist” refers to a molecule which intensifies or mimics the biological activity of HPEP. Agonists may include proteins, nucleic acids, carbohydrates, small molecules, or any other compound or composition which modulates the activity of HPEP either by directly interacting with HPEP or by acting on components of the biological pathway in which HPEP participates.

An “allelic variant” is an alternative form of the gene encoding HPEP. Allelic variants may result from at least one mutation in the nucleic acid sequence and may result in altered mRNAs or in polypeptides whose structure or function may or may not be altered. A gene may have none, one, or many allelic variants of its naturally occurring form. Common mutational changes which give rise to allelic variants are generally ascribed to natural deletions, additions, or substitutions of nucleotides. Each of these types of changes may occur alone, or in combination with the others, one or more times in a given sequence.

“Altered” nucleic acid sequences encoding HPEP include those sequences with deletions, insertions, or substitutions of different nucleotides, resulting in a polypeptide the same as HPEP or a polypeptide with at least one functional characteristic of HPEP. Included within this definition are polymorphisms which may or may not be readily detectable using a particular oligonucleotide probe of the polynucleotide encoding HPEP, and improper or unexpected hybridization to allelic variants, with a locus other than the normal chromosomal locus for the polynucleotide sequence encoding HPEP. The encoded protein may also be “altered,” and may contain deletions, insertions, or substitutions of amino acid residues which produce a silent change and result in a functionally equivalent HPEP. Deliberate amino acid substitutions may be made on the basis of similarity in polarity, charge, solubility, hydrophobicity, hydrophilicity, and/or the amphipathic nature of the residues, as long as the biological or immunological activity of HPEP is retained. For example, negatively charged amino acids may include aspartic acid and glutamic acid, and positively charged amino acids may include lysine and arginine. Amino acids with uncharged polar side chains having similar hydrophilicity values may include: asparagine and glutamine; and serine and threonine. Amino acids with uncharged side chains having similar hydrophilicity values may include: leucine, isoleucine, and valine; glycine and alanine; and phenylalanine and tyrosine.

The terms “amino acid” and “amino acid sequence” refer to an oligopeptide, peptide, polypeptide, or protein sequence, or a fragment of any of these, and to naturally occurring or synthetic molecules. Where “amino acid sequence” is recited to refer to an amino acid sequence of a naturally occurring protein molecule, “amino acid sequence” and like terms are not meant to limit the amino acid sequence to the complete native amino acid sequence associated with the recited protein molecule.

“Amplification” relates to the production of additional copies of a nucleic acid sequence. Amplification is generally carried out using polymerase chain reaction (PCR) technologies well known in the art.

The term “antagonist” refers to a molecule which inhibits or attenuates the biological activity of HPEP. Antagonists may include proteins such as antibodies, nucleic acids, carbohydrates, small molecules, or any other compound or composition which modulates the activity of HPEP either by directly interacting with HPEP or by acting on components of the biological pathway in which HPEP participates.

The term “antibody” refers to intact immunoglobulin molecules as well as to fragments thereof, such as Fab, F(ab′)₂, and Fv fragments, which are capable of binding an epitopic determinant. Antibodies that bind HPEP polypeptides can be prepared using intact polypeptides or using fragments containing small peptides of interest as the immunizing antigen. The polypeptide or oligopeptide used to immunize an animal (e.g., a mouse, a rat, or a rabbit) can be derived from the translation of RNA, or synthesized chemically, and can be conjugated to a carrier protein if desired. Commonly used carriers that are chemically coupled to peptides include bovine serum albumin, thyroglobulin, and keyhole limpet hemocyanin (KLH). The coupled peptide is then used to immunize the animal.

The term “antigenic determinant” refers to that region of a molecule (i.e., an epitope) that makes contact with a particular antibody. When a protein or a fragment of a protein is used to immunize a host animal, numerous regions of the protein may induce the production of antibodies which bind specifically to antigenic determinants (particular regions or three-dimensional structures on the protein). An antigenic determinant may compete with the intact antigen (i.e., the immunogen used to elicit the immune response) for binding to an antibody.

The term “antisense” refers to any composition containing a nucleic acid sequence which is complementary to the “sense” strand of a specific nucleic acid sequence. Antisense molecules may be produced by any method including synthesis or transcription. Once introduced into a cell, the complementary nucleotides combine with natural sequences produced by the cell to form duplexes and to block either transcription or translation. The designation “negative” or “minus” can refer to the antisense strand, and the designation “positive” or “plus” can refer to the sense strand.

The term “biologically active” refers to a protein having structural, regulatory, or biochemical functions of a naturally occurring molecule. Likewise, “immunologically active” refers to the capability of the natural, recombinant, or synthetic HPEP, or of any oligopeptide thereof, to induce a specific immune response in appropriate animals or cells and to bind with specific antibodies.

The terms “complementary” and “complementarity” refer to the natural binding of polynucleotides by base pairing. For example, the sequence “5′ A-G-T 3′” bonds to the complementary sequence “3′ T-C-A 5′.” Complementarity between two single-stranded molecules may be “partial,” such that only some of the nucleic acids bind, or it may be “complete,” such that total complementarity exists between the single stranded molecules. The degree of complementarity between nucleic acid strands has significant effects on the efficiency and strength of the hybridization between the nucleic acid strands. This is of particular importance in amplification reactions, which depend upon binding between nucleic acid strands, and in the design and use of peptide nucleic acid (PNA) molecules.

A “composition comprising a given polynucleotide sequence” and a “composition comprising a given amino acid sequence” refer broadly to any composition containing the given polynucleotide or amino acid sequence. The composition may comprise a dry formulation or an aqueous solution. Compositions comprising polynucleotide sequences encoding HPEP or fragments of HPEP may be employed as hybridization probes. The probes may be stored in freeze-dried form and may be associated with a stabilizing agent such as a carbohydrate. In hybridizations, the probe may be deployed in an aqueous solution containing salts (e.g., NaCl), detergents (e.g., sodium dodecyl sulfate; SDS), and other components (e.g., Denhardt's solution, dry milk, salmon sperm DNA, etc.).

“Consensus sequence” refers to a nucleic acid sequence which has been resequenced to resolve uncalled bases, extended using the XL-PCR kit (Perkin-Elmer, Norwalk CT) in the 5′ and/or the 3′ direction, and resequenced, or which has been assembled from the overlapping sequences of one or more Incyte Clones and, in some cases, one or more public domain ESTs, using a computer program for fragment assembly, such as the GELVIEW fragment assembly system (GCG, Madison Wis.). Some sequences have been both extended and assembled to produce the consensus sequence.

“Conservative amino acid substitutions” are those substitutions that, when made, least interfere with the properties of the original protein, i.e., the structure and especially the function of the protein is conserved and not significantly changed by such substitutions. The table below shows amino acids which may be substituted for an original amino acid in a protein and which are regarded as conservative amino acid substitutions.

| Original Residue | Conservative Substitution |
|---|---|
| Ala | Gly, Ser |
| Arg | His, Lys |
| Asn | Asp, Gln, His |
| Asp | Asn, Glu |
| Cys | Ala, Ser |
| Gln | Asn, Glu, His |
| Glu | Asp, Gln, His |
| Gly | Ala |
| His | Asn, Arg, Gln, Glu |
| Ile | Leu, Val |
| Leu | Ile, Val |
| Lys | Arg, Gln, Glu |
| Met | Leu, Ile |
| Phe | His, Met, Leu, Trp, Tyr |
| Ser | Cys, Thr |
| Thr | Ser, Val |
| Trp | Phe, Tyr |
| Tyr | His, Phe, Trp |
| Val | Ile, Leu, Thr |

Conservative amino acid substitutions generally maintain (a) the structure of the polypeptide backbone in the area of the substitution, for example, as a beta sheet or alpha helical conformation, (b) the charge or hydrophobicity of the molecule at the site of the substitution, and/or (c) the bulk of the side chain.

A “deletion” refers to a change in the amino acid or nucleotide sequence that results in the absence of one or more amino acid residues or nucleotides.

The term “derivative” refers to the chemical modification of a polypeptide sequence, or a polynucleotide sequence. Chemical modifications of a polynucleotide sequence can include, for example, replacement of hydrogen by an alkyl, acyl, hydroxyl, or amino group. A derivative polynucleotide encodes a polypeptide which retains at least one biological or immunological function of the natural molecule. A derivative polypeptide is one modified by glycosylation, pegylation, or any similar process that retains at least one biological or immunological function of the polypeptide from which it was derived.

A “fragment” is a unique portion of HPEP or the polynucleotide encoding HPEP which is identical in sequence to but shorter in length than the parent sequence. A fragment may comprise up to the entire length of the defined sequence, minus one nucleotide/amino acid residue. For example, a fragment may comprise from 5 to 1000 contiguous nucleotides or amino acid residues. A fragment used as a probe, primer, antigen, therapeutic molecule, or for other purposes, may be at least 5, 10, 15, 20, 25, 30, 40, 50, 60, 75, 100, 150, 250 or at least 500 contiguous nucleotides or amino acid residues in length. Fragments may be preferentially selected from certain regions of a molecule. For example, a polypeptide fragment may comprise a certain length of contiguous amino acids selected from the first 250 or 500 amino acids (or first 25% or 50% of a polypeptide) as shown in a certain defined sequence. Clearly these lengths are exemplary, and any length that is supported by the specification, including the Sequence Listing, tables, and figures, may be encompassed by the present embodiments.

A fragment of SEQ ID NO:19-36 comprises a region of unique polynucleotide sequence that specifically identifies SEQ ID NO:19-36, for example, as distinct from any other sequence in the same genome. A fragment of SEQ ID NO:19-36 is useful, for example, in hybridization and amplification technologies and in analogous methods that distinguish SEQ ID NO:19-36 from related polynucleotide sequences. The precise length of a fragment of SEQ ID NO:19-36 and the region of SEQ ID NO:19-36 to which the fragment corresponds are routinely determinable by one of ordinary skill in the art based on the intended purpose for the fragment.

A fragment of SEQ ID NO:1-18 is encoded by a fragment of SEQ ID NO:19-36. A fragment of SEQ ID NO:1-18 comprises a region of unique amino acid sequence that specifically identifies SEQ ID NO:1-18. For example, a fragment of SEQ ID NO:1-18 is useful as an immunogenic peptide for the development of antibodies that specifically recognize SEQ ID NO:1-18. The precise length of a fragment of SEQ ID NO:1-18 and the region of SEQ ID NO:1-18 to which the fragment corresponds are routinely determinable by one of ordinary skill in the art based on the intended purpose for the fragment.

The term “similarity” refers to a degree of complementarity. There may be partial similarity or complete similarity. The word “identity” may substitute for the word “similarity.” A partially complementary sequence that at least partially inhibits an identical sequence from hybridizing to a target nucleic acid is referred to as “substantially similar.” The inhibition of hybridization of the completely complementary sequence to the target sequence may be examined using a hybridization assay (Southern or northern blot, solution hybridization, and the like) under conditions of reduced stringency. A substantially similar sequence or hybridization probe will compete for and inhibit the binding of a completely similar (identical) sequence to the target sequence under conditions of reduced stringency. This is not to say that conditions of reduced stringency are such that non-specific binding is permitted, as reduced stringency conditions require that the binding of two sequences to one another be a specific (i.e., a selective) interaction. The absence of non-specific binding may be tested by the use of a second target sequence which lacks even a partial degree of complementarity (e.g., less than about 30% similarity or identity). In the absence of non-specific binding, the substantially similar sequence or probe will not hybridize to the second non-complementary target sequence.

The phrases “percent identity” and “% identity,” as applied to polynucleotide sequences, refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences.

Percent identity between polynucleotide sequences may be determined using the default parameters of the CLUSTAL V algorithm as incorporated into the MEGALIGN version 3.12e sequence alignment program. This program is part of the LASERGENE software package, a suite of molecular biological analysis programs (DNASTAR, Madison Wis.). CLUSTAL V is described in Higgins, D. G. and P. M. Sharp (1989) CABIOS 5:151-153 and in Higgins, D. G. et al. (1992) CABIOS 8:189-191. For pairwise alignments of polynucleotide sequences, the default parameters are set as follows: Ktuple=2, gap penalty=5, window=4, and “diagonals saved”=4. The “weighted” residue weight table is selected as the default. Percent identity is reported by CLUSTAL V as the “percent similarity” between aligned polynucleotide sequence pairs.

Alternatively, a suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403-410), which is available from several sources, including the NCBI, Bethesda, Md., and on the Internet at ncbi.nlm.nih.gov/BLAST/. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at ncbi.nlm.nih.gov/gorf/bl2.html. The “BLAST 2 Sequences” tool can be used for both blastn and blastp (discussed below). BLAST programs are commonly used with gap and other parameters set to default settings. For example, to compare two nucleotide sequences, one may use blastn with the “BLAST 2 Sequences” tool Version 2.0.9 (May 7, 1999) set at default parameters. Such default parameters may be, for example:

Matrix: BLOSUM62

Reward for match: 1

Penalty for mismatch: −2

Open Gap: 5 and Extension Gap: 2 penalties

Gap x drop-off: 50

Expect: 10

Word Size: 11

Filter: on

Percent identity may be measured over the length of an entire defined sequence, for example, as defined by a particular SEQ ID number, or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length supported by the sequences shown herein, in the tables, figures, or Sequence Listing, may be used to describe a length over which percentage identity may be measured.

Nucleic acid sequences that do not show a high degree of identity may nevertheless encode similar amino acid sequences due to the degeneracy of the genetic code. It is understood that changes in a nucleic acid sequence can be made using this degeneracy to produce multiple nucleic acid sequences that all encode substantially the same protein.

The phrases “percent identity” and “% identity,” as applied to polypeptide sequences, refer to the percentage of residue matches between at least two polypeptide sequences aligned using a standardized algorithm. Methods of polypeptide sequence alignment are well-known. Some alignment methods take into account conservative amino acid substitutions. Such conservative substitutions, explained in more detail above, generally preserve the hydrophobicity and acidity at the site of substitution, thus preserving the structure (and therefore function) of the polypeptide.

Percent identity between polypeptide sequences may be determined using the default parameters of the CLUSTAL V algorithm as incorporated into the MEGALIGN version 3.12e sequence alignment program (described and referenced above). For pairwise alignments of polypeptide sequences using CLUSTAL V, the default parameters are set as follows: Ktuple=1, gap penalty=3, window=5, and “diagonals saved”=5. The PAM250 matrix is selected as the default residue weight table. As with polynucleotide alignments, the percent identity is reported by CLUSTAL V as the “percent similarity” between aligned polypeptide sequence pairs.

Alternatively the NCBI BLAST software suite may be used. For example, for a pairwise comparison of two polypeptide sequences, one may use the “BLAST 2 Sequences” tool Version 2.0.9 (May 7, 1999) with blastp set at default parameters. Such default parameters may be, for example:

Matrix: BLOSUM62

Open Gap: 11 and Extension Gap: 1 penalties

Gap x drop-off: 50

Expect: 10

Word Size: 3

Filter: on

Percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

“Human artificial chromosomes” (HACs) are linear microchromosomes which may contain DNA sequences of about 6 kb to 10 Mb in size, and which contain all of the elements required for stable mitotic chromosome segregation and maintenance.

The term “humanized antibody” refers to antibody molecules in which the amino acid sequence in the non-antigen binding regions has been altered so that the antibody more closely resembles a human antibody, and still retains its original binding ability.

“Hybridization” refers to the process by which a polynucleotide strand anneals with a complementary strand through base pairing under defined hybridization conditions. Specific hybridization is an indication that two nucleic acid sequences share a high degree of identity. Specific hybridization complexes form under permissive annealing conditions and remain hybridized after the “washing” step(s). The washing step(s) is particularly important in determining the stringency of the hybridization process, with more stringent conditions allowing less non-specific binding, i.e., binding between pairs of nucleic acid strands that are not perfectly matched. Permissive conditions for annealing of nucleic acid sequences are routinely determinable by one of ordinary skill in the art and may be consistent among hybridization experiments, whereas wash conditions may be varied among experiments to achieve the desired stringency, and therefore hybridization specificity. Permissive annealing conditions occur, for example, at 68° C. in the presence of about 6× SSC, about 1% (w/v) SDS, and about 100 μg/ml denatured salmon sperm DNA.

Generally, stringency of hybridization is expressed, in part, with reference to the temperature under which the wash step is carried out. Generally, such wash temperatures are selected to be about 5° C. to 20° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. An equation for calculating Tₘ and conditions for nucleic acid hybridization are well known and can be found in Sambrook et al., 1989, Molecular Cloning: A Laboratory Manual, 2^{nd} ed., vol. 1-3, Cold Spring Harbor Press, Plainview N.Y.; specifically see volume 2, chapter 9.

High stringency conditions for hybridization between polynucleotides of the present invention include wash conditions of 68° C. in the presence of about 0.2× SSC and about 0.1% SDS, for 1 hour. Alternatively, temperatures of about 65° C., 60° C., 55° C., or 42° C. may be used. SSC concentration may be varied from about 0.1 to 2× SSC, with SDS being present at about 0.1%. Typically, blocking reagents are used to block non-specific hybridization. Such blocking reagents include, for instance, denatured salmon sperm DNA at about 100-200 μg/ml. Organic solvent, such as formamide at a concentration of about 35-50% v/v, may also be used under particular circumstances, such as for RNA:DNA hybridizations. Useful variations on these wash conditions will be readily apparent to those of ordinary skill in the art. Hybridization, particularly under high stringency conditions, may be suggestive of evolutionary similarity between the nucleotides. Such similarity is strongly indicative of a similar role for the nucleotides and their encoded polypeptides.

The term “hybridization complex” refers to a complex formed between two nucleic acid sequences by virtue of the formation of hydrogen bonds between complementary bases. A hybridization complex may be formed in solution (e.g., C₀t or R₀t analysis) or formed between one nucleic acid sequence present in solution and another nucleic acid sequence immobilized on a solid support (e.g., paper, membranes, filters, chips, pins or glass slides, or any other appropriate substrate to which cells or their nucleic acids have been fixed).

The words “insertion” and “addition” refer to changes in an amino acid or nucleotide sequence resulting in the addition of one or more amino acid residues or nucleotides, respectively.

“Immune response” can refer to conditions associated with inflammation, trauma, immune disorders, or infectious or genetic disease, etc. These conditions can be characterized by expression of various factors, e.g., cytokines, chemokines, and other signaling molecules, which may affect cellular and systemic defense systems.

The term “microarray” refers to an arrangement of distinct polynucleotides on a substrate.

The terms “element” and “array element” in a microarray context, refer to hybridizable polynucleotides arranged on the surface of a substrate.

The term “modulate” refers to a change in the activity of HPEP. For example, modulation may cause an increase or a decrease in protein activity, binding characteristics, or any other biological, functional, or immunological properties of HPEP.

The phrases “nucleic acid” and “nucleic acid sequence” refer to a nucleotide, oligonucleotide, polynucleotide, or any fragment thereof. These phrases also refer to DNA or RNA of genomic or synthetic origin which may be single-stranded or double-stranded and may represent the sense or the antisense strand, to peptide nucleic acid (PNA), or to any DNA-like or RNA-like material.

“Operably linked” refers to the situation in which a first nucleic acid sequence is placed in a functional relationship with the second nucleic acid sequence. For instance, a promoter is operably linked to a coding sequence if the promoter affects the transcription or expression of the coding sequence. Generally, operably linked DNA sequences may be in close proximity or contiguous and, where necessary to join two protein coding regions, in the same reading frame.

“Peptide nucleic acid” (PNA) refers to an antisense molecule or anti-gene agent which comprises an oligonucleotide of at least about 5 nucleotides in length linked to a peptide backbone of amino acid residues ending in lysine. The terminal lysine confers solubility to the composition. PNAs preferentially bind complementary single stranded DNA or RNA and stop transcript elongation, and may be pegylated to extend their lifespan in the cell.

“Probe” refers to nucleic acid sequences encoding HPEP, their complements, or fragments thereof, which are used to detect identical, allelic or related nucleic acid sequences. Probes are isolated oligonucleotides or polynucleotides attached to a detectable label or reporter molecule. Typical labels include radioactive isotopes, ligands, chemiluminescent agents, and enzymes. “Primers” are short nucleic acids, usually DNA oligonucleotides, which may be annealed to a target polynucleotide by complementary base-pairing. The primer may then be extended along the target DNA strand by a DNA polymerase enzyme. Primer pairs can be used for amplification (and identification) of a nucleic acid sequence, e.g., by the polymerase chain reaction (PCR).

Probes and primers as used in the present invention typically comprise at least 15 contiguous nucleotides of a known sequence. In order to enhance specificity, longer probes and primers may also be employed, such as probes and primers that comprise at least 20, 25, 30, 40, 50, 60, 70, 80, 90, 100, or at least 150 consecutive nucleotides of the disclosed nucleic acid sequences. Probes and primers may be considerably longer than these examples, and it is understood that any length supported by the specification, including the tables, figures, and Sequence Listing, may be used.

Methods for preparing and using probes and primers are described in the references, for example Sambrook et al., 1989, Molecular Cloning: A Laboratory Manual, 2^{nd} ed., vol. 1-3, Cold Spring Harbor Press, Plainview N.Y.; Ausubel et al., 1987, Current Protocols in Molecular Biology, Greene Publ. Assoc. & Wiley-Intersciences, New York N.Y.; Innis et al., 1990, PCR Protocols, A Guide to Methods and Applications, Academic Press, San Diego Calif. PCR primer pairs can be derived from a known sequence, for example, by using computer programs intended for that purpose such as Primer (Version 0.5, 1991, Whitehead Institute for Biomedical Research, Cambridge Mass.).

Oligonucleotides for use as primers are selected using software known in the art for such purpose. For example, OLIGO 4.06 software is useful for the selection of PCR primer pairs of up to 100 nucleotides each, and for the analysis of oligonucleotides and larger polynucleotides of up to 5,000 nucleotides from an input polynucleotide sequence of up to 32 kilobases. Similar primer selection programs have incorporated additional features for expanded capabilities. For example, the PrimOU primer selection program (available to the public from the Genome Center at University of Texas South West Medical Center, Dallas Tex.) is capable of choosing specific primers from megabase sequences and is thus useful for designing primers on a genome-wide scope. The Primer3 primer selection program (available to the public from the Whitehead Institute/MIT Center for Genome Research, Cambridge Mass.) allows the user to input a “mispriming library,” in which sequences to avoid as primer binding sites are user-specified. Primer3 is useful, in particular, for the selection of oligonucleotides for microarrays. (The source code for the latter two primer selection programs may also be obtained from their respective sources and modified to meet the user's specific needs.) The PrimeGen program (available to the public from the UK Human Genome Mapping Project Resource Centre, Cambridge UK) designs primers based on multiple sequence alignments, thereby allowing selection of primers that hybridize to either the most conserved or least conserved regions of aligned nucleic acid sequences. Hence, this program is useful for identification of both unique and conserved oligonucleotides and polynucleotide fragments. The oligonucleotides and polynucleotide fragments identified by any of the above selection methods are useful in hybridization technologies, for example, as PCR or sequencing primers, microarray elements, or specific probes to identify fully or partially complementary polynucleotides in a sample of nucleic acids. Methods of oligonucleotide selection are not limited to those described above.

A “recombinant nucleic acid” is a sequence that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques such as those described in Sambrook, supra. The term recombinant includes nucleic acids that have been altered solely by addition, substitution, or deletion of a portion of the nucleic acid. Frequently, a recombinant nucleic acid may include a nucleic acid sequence operably linked to a promoter sequence. Such a recombinant nucleic acid may be part of a vector that is used, for example, to transform a cell.

Alternatively, such recombinant nucleic acids may be part of a viral vector, e.g., based on a vaccinia virus, that could be use to vaccinate a mammal wherein the recombinant nucleic acid is expressed, inducing a protective immunological response in the mammal.

The term “sample” is used in its broadest sense. A sample suspected of containing nucleic acids encoding HPEP, or fragments thereof, or HPEP itself, may comprise a bodily fluid; an extract from a cell, chromosome, organelle, or membrane isolated from a cell; a cell; genomic DNA, RNA, or cDNA, in solution or bound to a substrate; a tissue; a tissue print; etc.

The terms “specific binding” and “specifically binding” refer to that interaction between a protein or peptide and an agonist, an antibody, an antagonist, a small molecule, or any natural or synthetic binding composition. The interaction is dependent upon the presence of a particular structure of the protein, e.g., the antigenic determinant or epitope, recognized by the binding molecule. For example, if an antibody is specific for epitope “A,” the presence of a polypeptide containing the epitope A, or the presence of free unlabeled A, in a reaction containing free labeled A and the antibody will reduce the amount of labeled A that binds to the antibody.

The term “substantially purified” refers to nucleic acid or amino acid sequences that are removed from their natural environment and are isolated or separated, and are at least 60% free, preferably at least 75% free, and most preferably at least 90% free from other components with which they are naturally associated.

A “substitution” refers to the replacement of one or more amino acids or nucleotides by different amino acids or nucleotides, respectively.

“Substrate” refers to any suitable rigid or semi-rigid support including membranes, filters, chips, slides, wafers, fibers, magnetic or nonmagnetic beads, gels, tubing, plates, polymers, microparticles and capillaries. The substrate can have a variety of surface forms, such as wells, trenches, pins, channels and pores, to which polynucleotides or polypeptides are bound.

“Transformation” describes a process by which exogenous DNA enters and changes a recipient cell. Transformation may occur under natural or artificial conditions according to various methods well known in the art, and may rely on any known method for the insertion of foreign nucleic acid sequences into a prokaryotic or eukaryotic host cell. The method for transformation is selected based on the type of host cell being transformed and may include, but is not limited to, viral infection, electroporation, heat shock, lipofection, and particle bombardment. The term “transformed” cells includes stably transformed cells in which the inserted DNA is capable of replication either as an autonomously replicating plasmid or as part of the host chromosome, as well as transiently transformed cells which express the inserted DNA or RNA for limited periods of time.

A “variant” of a particular nucleic acid sequence is defined as a nucleic acid sequence having at least 40% sequence identity to the particular nucleic acid sequence over a certain length of one of the nucleic acid sequences using blastn with the “BLAST 2 Sequences” tool Version 2.0.9 (May 7, 1999) set at default parameters. Such a pair of nucleic acids may show, for example, at least 50%, at least 60%, at least 70%, at least 80%, at least 85%, at least 90%, at least 95% or at least 98% or greater sequence identity over a certain defined length. A variant may be described as, for example, an “allelic” (as defined above), “splice,” “species,” or “polymorphic” variant. A splice variant may have significant identity to a reference molecule, but will generally have a greater or lesser number of polynucleotides due to alternate splicing of exons during mRNA processing. The corresponding polypeptide may possess additional functional domains or lack domains that are present in the reference molecule. Species variants are polynucleotide sequences that vary from one species to another. The resulting polypeptides generally will have significant amino acid identity relative to each other. A polymorphic variant is a variation in the polynucleotide sequence of a particular gene between individuals of a given species. Polymorphic variants also may encompass “single nucleotide polymorphisms” (SNPs) in which the polynucleotide sequence varies by one nucleotide base. The presence of SNPs may be indicative of, for example, a certain population, a disease state, or a propensity for a disease state.

A “variant” of a particular polypeptide sequence is defined as a polypeptide sequence having at least 40% sequence identity to the particular polypeptide sequence over a certain length of one of the polypeptide sequences using blastp with the “BLAST 2 Sequences” tool Version 2.0.9 (May 7, 1999) set at default parameters. Such a pair of polypeptides may show, for example, at least 50%, at least 60%, at least 70%, at least 80%, at least 90%, at least 95%, or at least 98% or greater sequence identity over a certain defined length of one of the polypeptides.

The Invention

The invention is based on the discovery of new human peptidases (HPEP), the polynucleotides encoding HPEP, and the use of these compositions for the diagnosis, treatment, or prevention of cell proliferative, autoimmune/inflammatory, and metabolic disorders.

Table 1 lists the Incyte clones used to assemble full length nucleotide sequences encoding HPEP. Columns 1 and 2 show the sequence identification numbers (SEQ ID NOs) of the polypeptide and nucleotide sequences, respectively. Column 3 shows the clone IDs of the Incyte clones in which nucleic acids encoding each HPEP were identified, and column 4 shows the cDNA libraries from which these clones were isolated. Column 5 shows Incyte clones and their corresponding cDNA libraries. Clones for which cDNA libraries are not indicated were derived from pooled cDNA libraries. The Incyte clones in column 5 were used to assemble the consensus nucleotide sequence of each HPEP and are useful as fragments in hybridization technologies.

The columns of Table 2 show various properties of each of the polypeptides of the invention: column 1 references the SEQ ID NO; column 2 shows the number of amino acid residues in each polypeptide; column 3 shows potential phosphorylation sites; column 4 shows potential glycosylation sites; column 5 shows the amino acid residues comprising signature sequences and motifs; column 6 shows homologous sequences as identified by BLAST analysis; and column 7 shows analytical methods and in some cases, searchable databases to which the analytical methods were applied. The methods of column 7 were used to characterize each polypeptide through sequence homology and protein motifs.

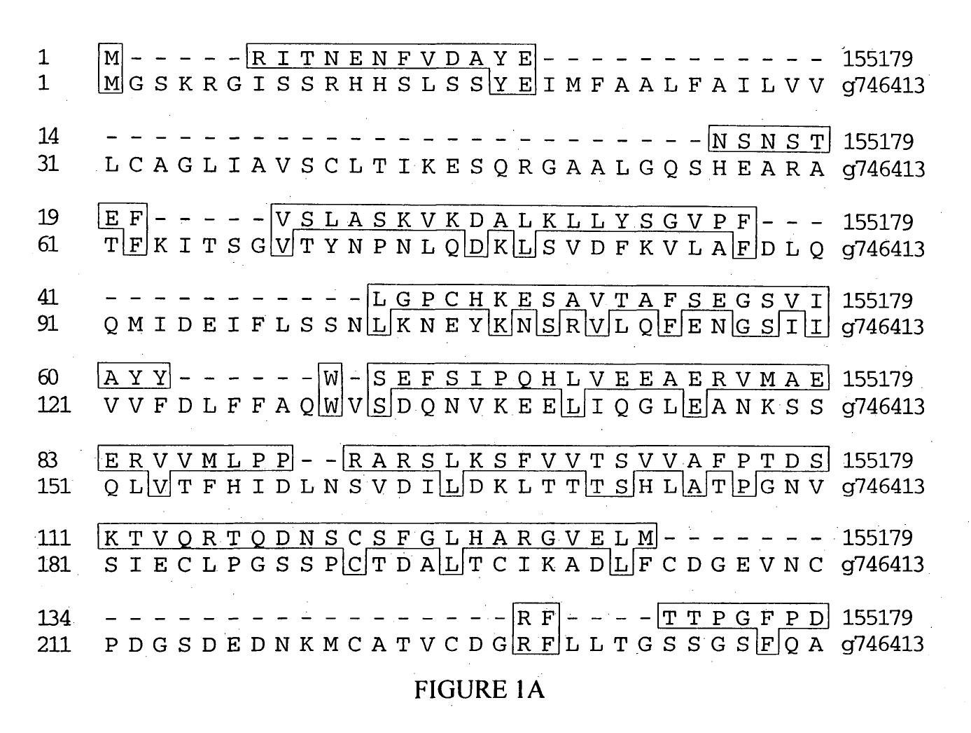

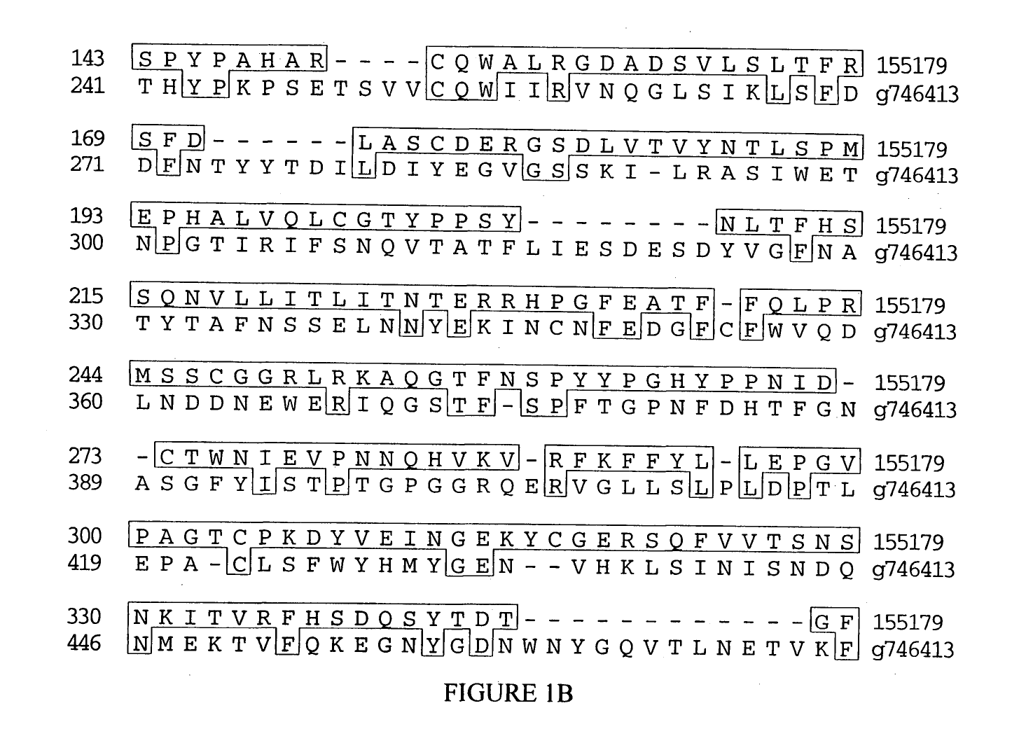

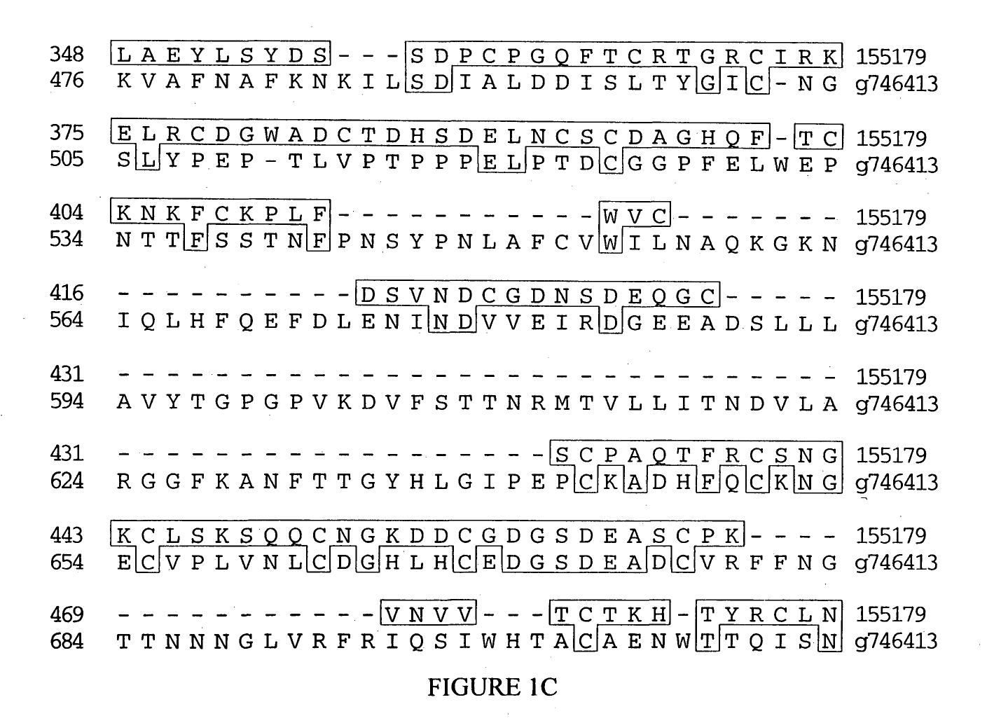

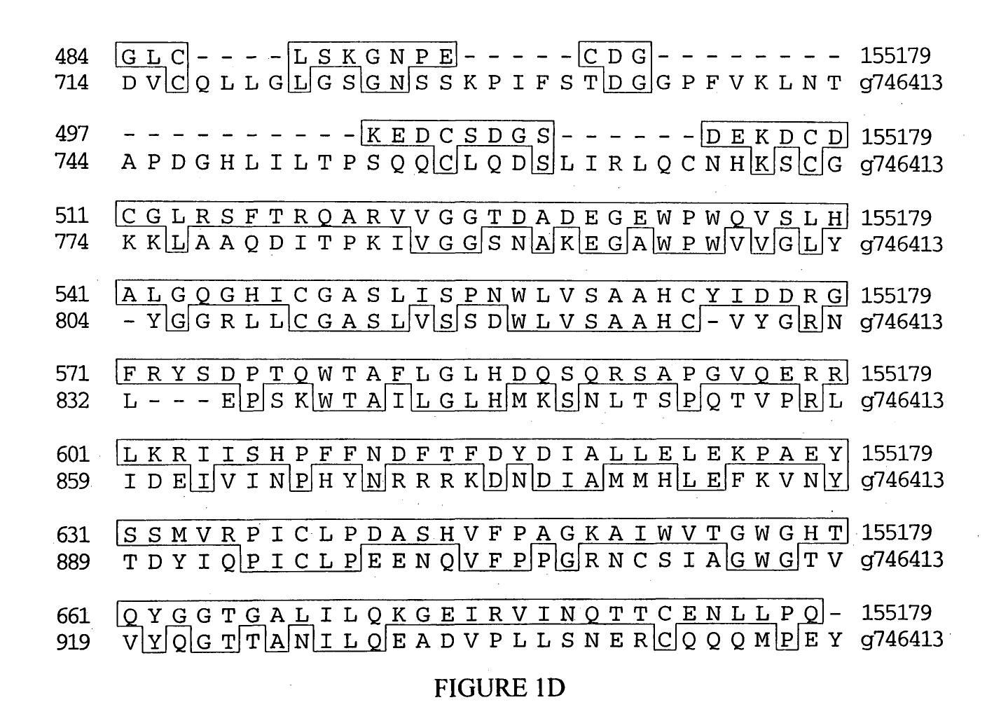

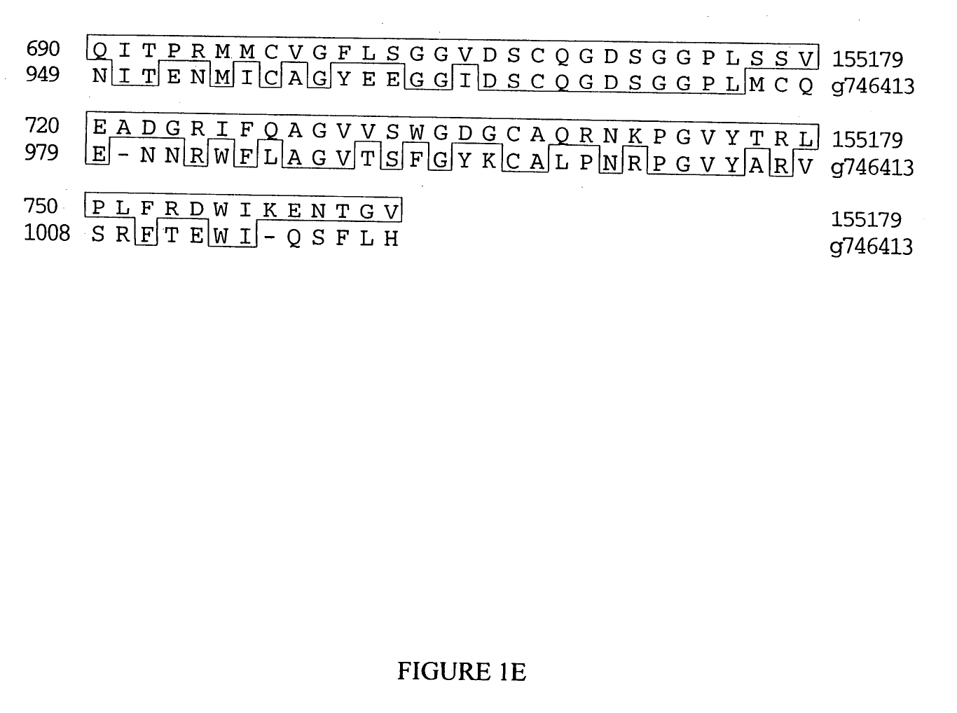

As shown in FIGS. 1A, 1B, 1C, 1D, and 1E, HPEP-1 has chemical and structural similarity with human enterokinase (GI 746413; SEQ ID NO:37). In particular, HPEP-1 and human enterokinase share 21% identity.

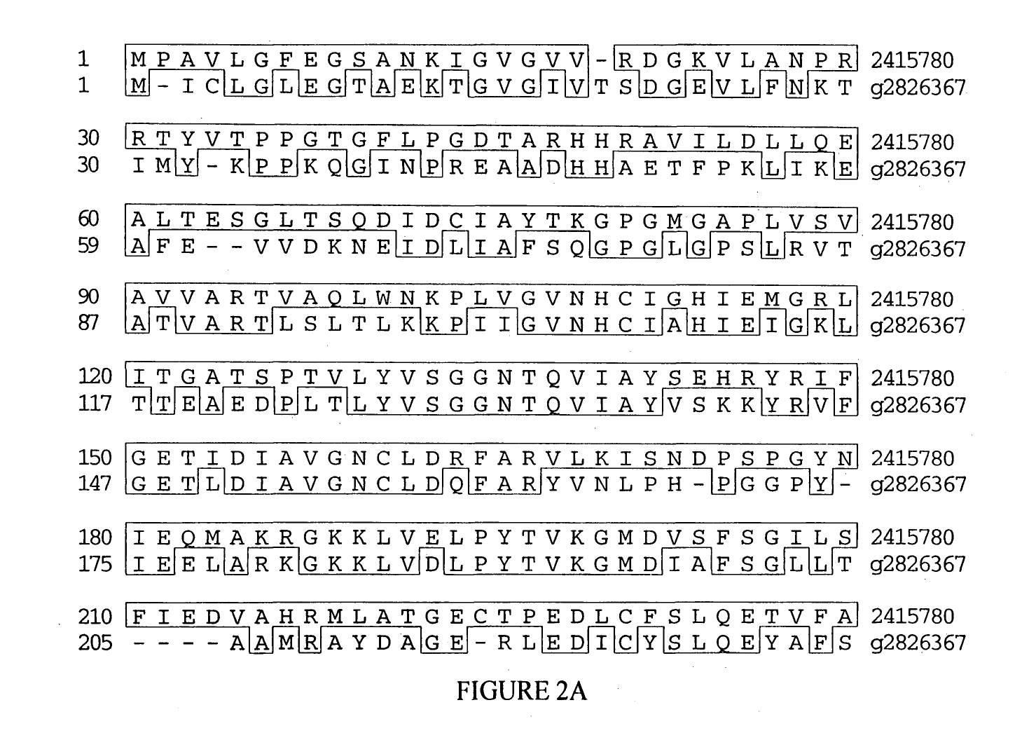

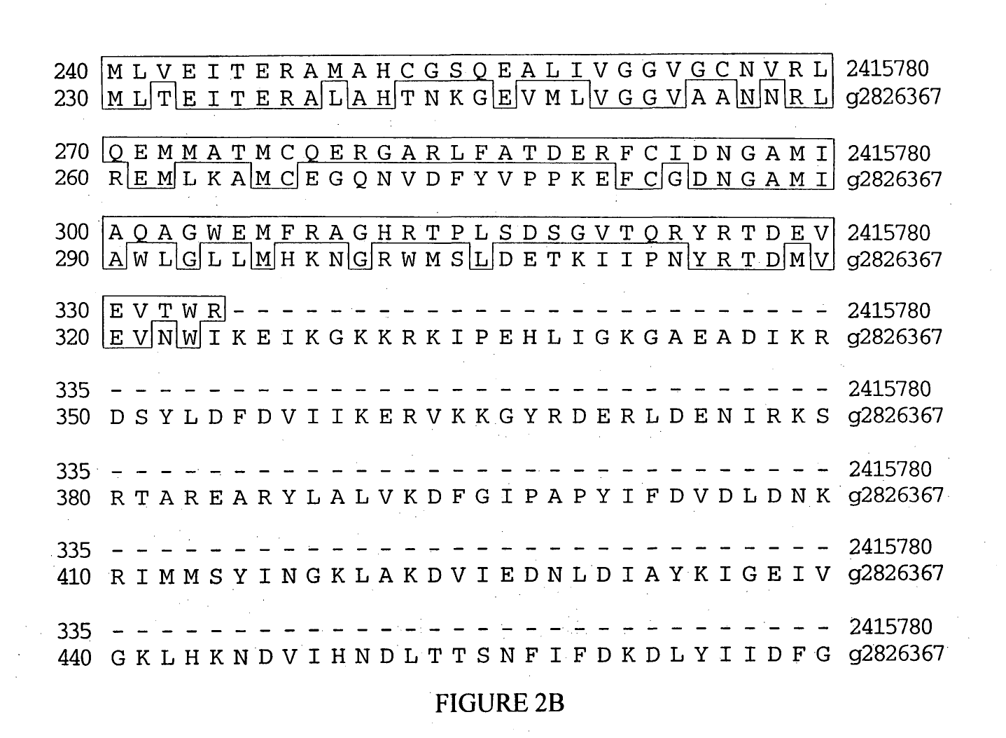

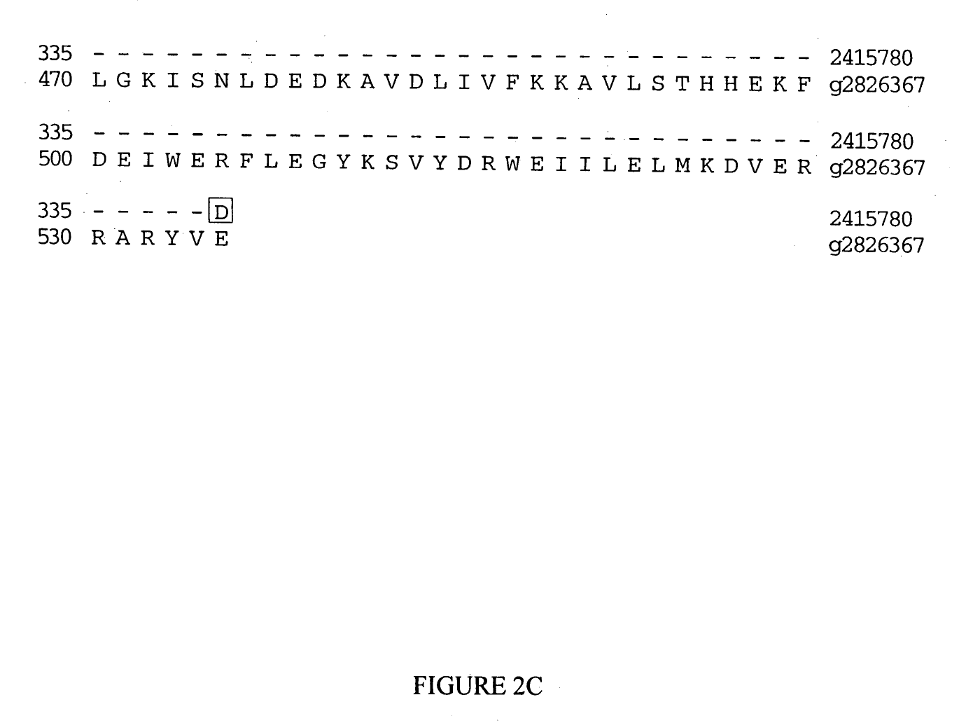

As shown in FIGS. 2A, 2B, and 2C, HPEP-2 has chemical and structural similarity with Methanococcus iannaschii o-sialoglycoprotein endopeptidase (GI 2826367; SEQ ID NO:38). In particular, HPEP-2 and Methanococcus jannaschii o-sialoglycoprotein endopeptidase share 44% identity.

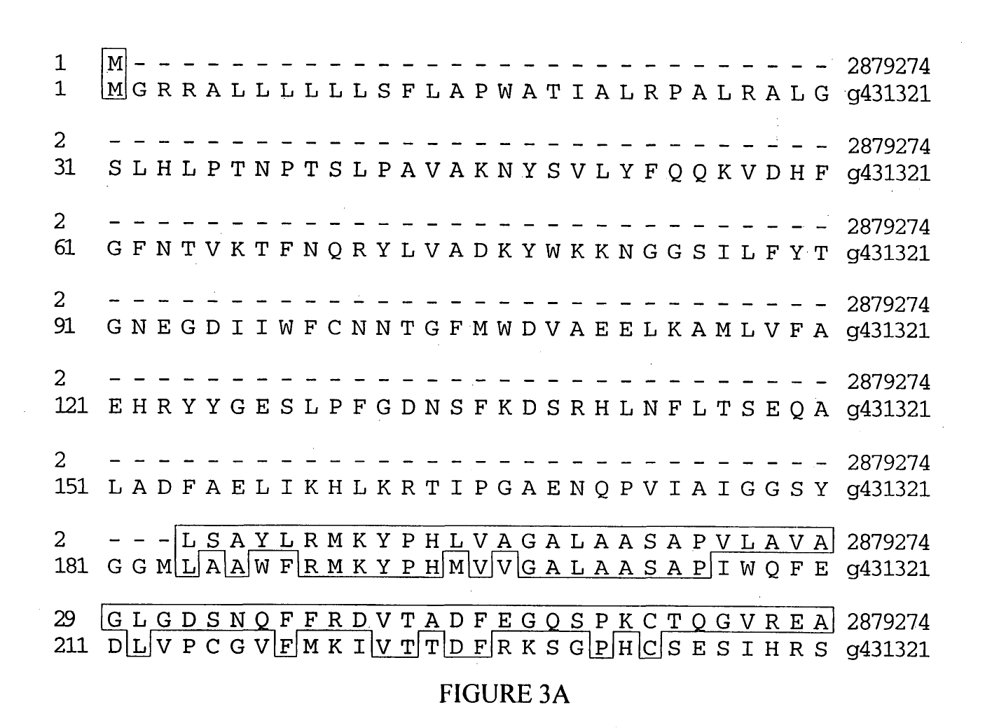

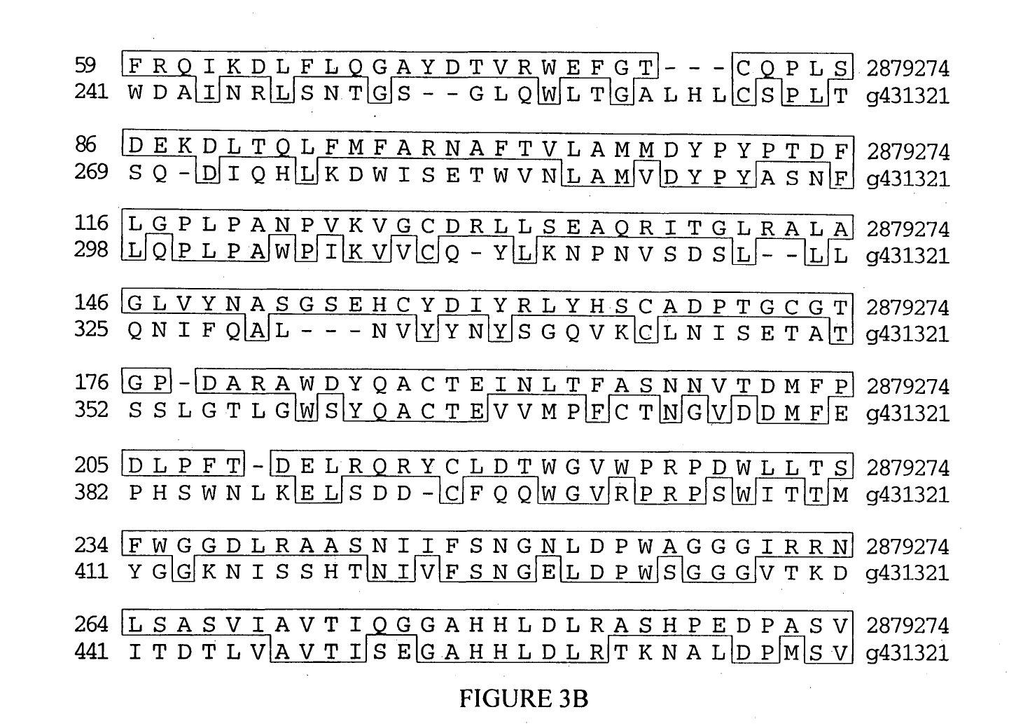

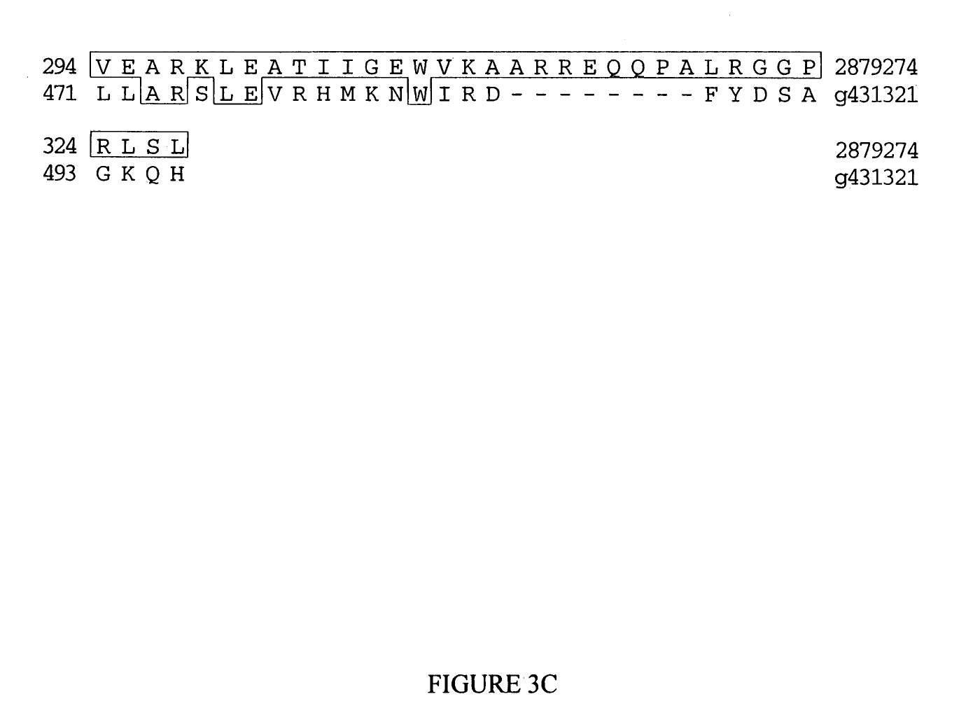

As shown in FIGS. 3A, 3B, and 3C, HPEP-3 has chemical and structural similarity with human prolylcarboxypeptidase (GI 431321; SEQ ID NO:39). In particular, HPEP-3 and human prolylcarboxypeptidase share 33% identity.

The columns of Table 3 show the tissue-specificity and diseases, disorders, or conditions associated with nucleotide sequences encoding HPEP. The first column of Table 3 lists the nucleotide SEQ ID NOs. Column 2 lists fragments of the nucleotide sequences of column 1. These fragments are useful, for example, in hybridization or amplification technologies to identify SEQ ID NO:19-36 and to distinguish between SEQ ID NO:19-36 and related polynucleotide sequences. The polypeptides encoded by these fragments are useful, for example, as immunogenic peptides. Column 3 lists tissue categories which express HPEP as a fraction of total tissues expressing HPEP. Column 4 lists diseases, disorders, or conditions associated with those tissues expressing HPEP as a fraction of total tissues expressing HPEP. Of particular note is the expression of SEQ ID NO:28 in tissues associated with inflammation and the immune response. Column 5 lists the vectors used to subclone each cDNA library.

Northern analysis shows the expression of SEQ ID NO:19 in various libraries, at least 66% of which are associated with cell proliferation and at least 31% of which are associated with inflammation and immune response. Of particular note is the expression of HPEP-1 in gastrointestinal tissues (33%), reproductive tissues (28%), and hematopoietic/immune tissues (28%).

Northern analysis shows the expression of SEQ ID NO:20 in various libraries, at least 59% of which are associated with cell proliferation and at least 43% of which are associated with inflammation and immune response. Of particular note is the expression of HPEP-2 in reproductive tissues (21%), hematopoietic/immune tissues (20%), and nervous tissues (19%).

Northern analysis shows the expression of SEQ ID NO:21 in various libraries, at least 61% of which are associated with cell proliferation and at least 34% of which are associated with inflammation and immune response. Of particular note is the expression of HPEP-3 in reproductive tissues (30%), nervous tissues (18%), and gastrointestinal tissues (12%).

The columns of Table 4 show descriptions of the tissues used to construct the cDNA libraries from which cDNA clones encoding HPEP were isolated. Column 1 references the nucleotide SEQ ID NOs, column 2 shows the cDNA libraries from which these clones were isolated, and column 3 shows the tissue origins and other descriptive information relevant to the cDNA libraries in column 2.

SEQ ID NO:30 maps to chromosome 17 within the interval from 75.70 to 83.90 centiMorgans. This interval also contains a gene associated with hepatic leukemia and estrogen response. SEQ ID NO:32 maps to chromosome 7 within the interval from 78.90 to 79.60 centiMorgans.

The invention also encompasses HPEP variants. A preferred HPEP variant is one which has at least about 80%, or alternatively at least about 90%, or even at least about 95% amino acid sequence identity to the HPEP amino acid sequence, and which contains at least one functional or structural characteristic of HPEP.

The invention also encompasses polynucleotides which encode HPEP. In a particular embodiment, the invention encompasses a polynucleotide sequence comprising a sequence selected from the group consisting of SEQ ID NO:19-36, which encodes HPEP.

The invention also encompasses a variant of a polynucleotide sequence encoding HPEP. In particular, such a variant polynucleotide sequence will have at least about 85%, or alternatively at least about 90%, or even at least about 95% polynucleotide sequence identity to the polynucleotide sequence encoding HPEP. A particular aspect of the invention encompasses a variant of a polynucleotide sequence comprising a sequence selected from the group consisting of SEQ ID NO:19-36 which has at least about 85%, or alternatively at least about 90%, or even at least about 95% polynucleotide sequence identity to a nucleic acid sequence selected from the group consisting of SEQ ID NO:19-36. Any one of the polynucleotide variants described above can encode an amino acid sequence which contains at least one functional or structural characteristic of HPEP.

It will be appreciated by those skilled in the art that as a result of the degeneracy of the genetic code, a multitude of polynucleotide sequences encoding HPEP, some bearing minimal similarity to the polynucleotide sequences of any known and naturally occurring gene, may be produced. Thus, the invention contemplates each and every possible variation of polynucleotide sequence that could be made by selecting combinations based on possible codon choices. These combinations are made in accordance with the standard triplet genetic code as applied to the polynucleotide sequence of naturally occurring HPEP, and all such variations are to be considered as being specifically disclosed.

Although nucleotide sequences which encode HPEP and its variants are generally capable of hybridizing to the nucleotide sequence of the naturally occurring HPEP under appropriately selected conditions of stringency, it may be advantageous to produce nucleotide sequences encoding HPEP or its derivatives possessing a substantially different codon usage, e.g., inclusion of non-naturally occurring codons. Codons may be selected to increase the rate at which expression of the peptide occurs in a particular prokaryotic or eukaryotic host in accordance with the frequency with which particular codons are utilized by the host. Other reasons for substantially altering the nucleotide sequence encoding HPEP and its derivatives without altering the encoded amino acid sequences include the production of RNA transcripts having more desirable properties, such as a greater half-life, than transcripts produced from the naturally occurring sequence.

The invention also encompasses production of DNA sequences which encode HPEP and HPEP derivatives, or fragments thereof, entirely by synthetic chemistry. After production, the synthetic sequence may be inserted into any of the many available expression vectors and cell systems using reagents well known in the art. Moreover, synthetic chemistry may be used to introduce mutations into a sequence encoding HPEP or any fragment thereof.

Also encompassed by the invention are polynucleotide sequences that are capable of hybridizing to the claimed polynucleotide sequences, and, in particular, to those shown in SEQ ID NO:19-36 and fragments thereof under various conditions of stringency. (See, e.g., Wahl, G. M. and S. L. Berger (1987) Methods Enzymol. 152:399-407; Kimmel, A. R. (1987) Methods Enzymol. 152:507-511.) Hybridization conditions, including annealing and wash conditions, are described in “Definitions.”

Methods for DNA sequencing are well known in the art and may be used to practice any of the embodiments of the invention. The methods may employ such enzymes as the Klenow fragment of DNA polymerase I, SEQUENASE (US Biochemical, Cleveland Ohio), Taq polymerase (Perkin-Elmer), thermostable T7 polymerase (Amersham Pharmacia Biotech, Piscataway N.J.), or combinations of polymerases and proofreading exonucleases such as those found in the ELONGASE amplification system (Life Technologies, Gaithersburg Md.). Preferably, sequence preparation is automated with machines such as the MICROLAB 2200 liquid transfer system (Hamilton, Reno Nev.), PTC200 thermal cycler (MJ Research, Watertown Mass.) and ABI CATALYST 800 thermal cycler (Perkin-Elmer). Sequencing is then carried out using either the ABI 373 or 377 DNA sequencing system (Perkin-Elmer), the MEGABACE 1000 DNA sequencing system (Molecular Dynamics, Sunnyvale Calif.), or other systems known in the art. The resulting sequences are analyzed using a variety of algorithms which are well known in the art. (See, e.g., Ausubel, F. M. (1997) Short Protocols in Molecular Biology, John Wiley & Sons, New York N.Y., unit 7.7; Meyers, R. A. (1995) Molecular Biology and Biotechnology, Wiley VCH, New York N.Y., pp. 856-853.)

The nucleic acid sequences encoding HPEP may be extended utilizing a partial nucleotide sequence and employing various PCR-based methods known in the art to detect upstream sequences, such as promoters and regulatory elements. For example, one method which may be employed, restriction-site PCR, uses universal and nested primers to amplify unknown sequence from genomic DNA within a cloning vector. (See, e.g., Sarkar, G. (1993) PCR Methods Applic. 2:318-322.) Another method, inverse PCR, uses primers that extend in divergent directions to amplify unknown sequence from a circularized template. The template is derived from restriction fragments comprising a known genomic locus and surrounding sequences. (See, e.g., Triglia, T. et al. (1988) Nucleic Acids Res. 16:8186.) A third method, capture PCR, involves PCR amplification of DNA fragments adjacent to known sequences in human and yeast artificial chromosome DNA. (See, e.g., Lagerstrom, M. et al. (1991) PCR Methods Applic. 1:111-119.) In this method, multiple restriction enzyme digestions and ligations may be used to insert an engineered double-stranded sequence into a region of unknown sequence before performing PCR. Other methods which may be used to retrieve unknown sequences are known in the art. (See, e.g., Parker, J. D. et al. (1991) Nucleic Acids Res. 19:3055-3060). Additionally, one may use PCR, nested primers, and PROMOTERFINDER libraries (Clontech, Palo Alto Calif.) to walk genomic DNA. This procedure avoids the need to screen libraries and is useful in finding intron/exon junctions. For all PCR-based methods, primers may be designed using commercially available software, such as OLIGO 4.06 Primer Analysis software (National Biosciences, Plymouth Minn.) or another appropriate program, to be about 22 to 30 nucleotides in length, to have a GC content of about 50% or more, and to anneal to the template at temperatures of about 68° C. to 72° C.

When screening for full-length cDNAs, it is preferable to use libraries that have been size-selected to include larger cDNAs. In addition, random-primed libraries, which often include sequences containing the 5′ regions of genes, are preferable for situations in which an oligo d(T) library does not yield a full-length cDNA. Genomic libraries may be useful for extension of sequence into 5′ non-transcribed regulatory regions.

Capillary electrophoresis systems which are commercially available may be used to analyze the size or confirm the nucleotide sequence of sequencing or PCR products. In particular, capillary sequencing may employ flowable polymers for electrophoretic separation, four different nucleotide-specific, laser-stimulated fluorescent dyes, and a charge coupled device camera for detection of the emitted wavelengths. Output/light intensity may be converted to electrical signal using appropriate software (e.g., GENOTYPER and SEQUENCE NAVIGATOR, Perkin-Elmer), and the entire process from loading of samples to computer analysis and electronic data display may be computer controlled. Capillary electrophoresis is especially preferable for sequencing small DNA fragments which may be present in limited amounts in a particular sample.

In another embodiment of the invention, polynucleotide sequences or fragments thereof which encode HPEP may be cloned in recombinant DNA molecules that direct expression of HPEP, or fragments or functional equivalents thereof, in appropriate host cells. Due to the inherent degeneracy of the genetic code, other DNA sequences which encode substantially the same or a functionally equivalent amino acid sequence may be produced and used to express HPEP.

The nucleotide sequences of the present invention can be engineered using methods generally known in the art in order to alter HPEP-encoding sequences for a variety of purposes including, but not limited to, modification of the cloning, processing, and/or expression of the gene product. DNA shuffling by random fragmentation and PCR reassembly of gene fragments and synthetic oligonucleotides may be used to engineer the nucleotide sequences. For example, oligonucleotide-mediated site-directed mutagenesis may be used to introduce mutations that create new restriction sites, alter glycosylation patterns, change codon preference, produce splice variants, and so forth.

The nucleotides of the present invention may be subjected to DNA shuffling techniques such as MOLECULARBREEDING (Maxygen Inc., Santa Clara Calif.; described in U.S. Pat. No. 5,837,458; Chang, C. -C. et al. (1999) Nat. Biotechnol. 17:793-797; Christians, F. C. et al. (1999) Nat. Biotechnol. 17:259-264; and Crameri, A. et al. (1996) Nat. Biotechnol. 14:315-319) to alter or improve the biological properties of HPEP, such as its biological or enzymatic activity or its ability to bind to other molecules or compounds. DNA shuffling is a process by which a library of gene variants is produced using PCR-mediated recombination of gene fragments. The library is then subjected to selection or screening procedures that identify those gene variants with the desired properties. These preferred variants may then be pooled and further subjected to recursive rounds of DNA shuffling and selection/screening. Thus, genetic diversity is created through “artificial” breeding and rapid molecular evolution. For example, fragments of a single gene containing random point mutations may be recombined, screened, and then reshuffled until the desired properties are optimized. Alternatively, fragments of a given gene may be recombined with fragments of homologous genes in the same gene family, either from the same or different species, thereby maximizing the genetic diversity of multiple naturally occurring genes in a directed and controllable manner.

In another embodiment, sequences encoding HPEP may be synthesized, in whole or in part, using chemical methods well known in the art. (See, e.g., Caruthers, M. H. et al. (1980) Nucleic Acids Symp. Ser. 7:215-223; and Horn, T. et al. (1980) Nucleic Acids Symp. Ser. 7:225-232.) Alternatively, HPEP itself or a fragment thereof may be synthesized using chemical methods. For example, peptide synthesis can be performed using various solid-phase techniques. (See, e.g., Roberge, J. Y. et al. (1995) Science 269:202-204.) Automated synthesis may be achieved using the ABI 431A peptide synthesizer (Perkin-Elmer). Additionally, the amino acid sequence of HPEP, or any part thereof, may be altered during direct synthesis and/or combined with sequences from other proteins, or any part thereof, to produce a variant polypeptide.

The peptide may be substantially purified by preparative high performance liquid chromatography. (See, e.g., Chiez, R. M. and F. Z. Regnier (1990) Methods Enzymol. 182:392-421.) The composition of the synthetic peptides may be confirmed by amino acid analysis or by sequencing. (See, e.g., Creighton, T. (1984) Proteins, Structures and Molecular Properties, WH Freeman, New York N.Y.)

In order to express a biologically active HPEP, the nucleotide sequences encoding HPEP or derivatives thereof may be inserted into an appropriate expression vector, i.e., a vector which contains the necessary elements for transcriptional and translational control of the inserted coding sequence in a suitable host. These elements include regulatory sequences, such as enhancers, constitutive and inducible promoters, and 5′ and 3′ untranslated regions in the vector and in polynucleotide sequences encoding HPEP. Such elements may vary in their strength and specificity. Specific initiation signals may also be used to achieve more efficient translation of sequences encoding HPEP. Such signals include the ATG initiation codon and adjacent sequences, e.g. the Kozak sequence. In cases where sequences encoding HPEP and its initiation codon and upstream regulatory sequences are inserted into the appropriate expression vector, no additional transcriptional or translational control signals may be needed. However, in cases where only coding sequence, or a fragment thereof, is inserted, exogenous translational control signals including an in-frame ATG initiation codon should be provided by the vector. Exogenous translational elements and initiation codons may be of various origins, both natural and synthetic. The efficiency of expression may be enhanced by the inclusion of enhancers appropriate for the particular host cell system used. (See, e.g., Scharf, D. et al. (1994) Results Probi. Cell Differ. 20:125-162.)

Methods which are well known to those skilled in the art may be used to construct expression vectors containing sequences encoding HPEP and appropriate transcriptional and translational control elements. These methods include in vitro recombinant DNA techniques, synthetic techniques, and in vivo genetic recombination. (See, e.g., Sambrook, J. et al. (1989) Molecular Cloning, A Laboratory Manual, Cold Spring Harbor Press, Plainview N.Y., ch. 4, 8, and 16-17; Ausubel, F. M. et al. (1995) Current Protocols in Molecular Biology, John Wiley & Sons, New York N.Y., ch. 9, 13, and 16.).

A variety of expression vector/host systems may be utilized to contain and express sequences encoding HPEP. These include, but are not limited to, microorganisms such as bacteria transformed with recombinant bacteriophage, plasmid, or cosmid DNA expression vectors; yeast transformed with yeast expression vectors; insect cell systems infected with viral expression vectors (e.g., baculovirus); plant cell systems transformed with viral expression vectors (e.g., cauliflower mosaic virus, CaMV, or tobacco mosaic virus, TMV) or with bacterial expression vectors (e.g., Ti or pBR322 plasmids); or animal cell systems. The invention is not limited by the host cell employed.

In bacterial systems, a number of cloning and expression vectors may be selected depending upon the use intended for polynucleotide sequences encoding HPEP. For example, routine cloning, subcloning, and propagation of polynucleotide sequences encoding HPEP can be achieved using a multifunctional E. coli vector such as PBLUESCRIPT (Stratagene, La Jolla Calif.) or PSPORT1 plasmid (Life Technologies). Ligation of sequences encoding HPEP into the vector's multiple cloning site disrupts the lacZ gene, allowing a calorimetric screening procedure for identification of transformed bacteria containing recombinant molecules. In addition, these vectors may be useful for in vitro transcription, dideoxy sequencing, single strand rescue with helper phage, and creation of nested deletions in the cloned sequence. (See, e.g., Van Heeke, G. and S. M. Schuster (1989) J. Biol. Chem. 264:5503-5509.) When large quantities of HPEP are needed, e.g. for the production of antibodies, vectors which direct high level expression of HPEP may be used. For example, vectors containing the strong, inducible T5 or T7 bacteriophage promoter may be used.

Yeast expression systems may be used for production of HPEP. A number of vectors containing constitutive or inducible promoters, such as alpha factor, alcohol oxidase, and PGH promoters, may be used in the yeast Saccharomyces cerevisiae or Pichia pastoris. In addition, such vectors direct either the secretion or intracellular retention of expressed proteins and enable integration of foreign sequences into the host genome for stable propagation. (See, e.g., Ausubel, 1995, supra; Bitter, G. A. et al. (1987) Methods Enzymol. 153:516-544; and Scorer, C. A. et al. (1994) Bio/Technology 12:181-184.)

Plant systems may also be used for expression of HPEP. Transcription of sequences encoding HPEP may be driven viral promoters, e.g., the 35S and 19S promoters of CaMV used alone or in combination with the omega leader sequence from TMV (Takamatsu, N. (1987) EMBO J. 6:307-311). Alternatively, plant promoters such as the small subunit of RUBISCO or heat shock promoters may be used. (See, e.g., Coruzzi, G. et al. (1984) EMBO J. 3:1671-1680; Broglie, R. et al. (1984) Science 224:838-843; and Winter, J. et al. (1991) Results Probl. Cell Differ. 17:85-105.) These constructs can be introduced into plant cells by direct DNA transformation or pathogen-mediated transfection. (See, e.g., The McGraw Hill Yearbook of Science and Technology (1992) McGraw Hill, New York N.Y., pp. 191-196.)

In mammalian cells, a number of viral-based expression systems may be utilized. In cases where an adenovirus is used as an expression vector, sequences encoding HPEP may be ligated into an adenovirus transcription/translation complex consisting of the late promoter and tripartite leader sequence. Insertion in a non-essential El or E3 region of the viral genome may be used to obtain infective virus which expresses HPEP in host cells. (See, e.g., Logan, J. and T. Shenk (1984) Proc. Natl. Acad. Sci. USA 81:3655-3659.) In addition, transcription enhancers, such as the Rous sarcoma virus (RSV) enhancer, may be used to increase expression in mammalian host cells. SV40 or EBV-based vectors may also be used for high-level protein expression.

Human artificial chromosomes (HACs) may also be employed to deliver larger fragments of DNA than can be contained in and expressed from a plasmid. HACs of about 6 kb to 10 Mb are constructed and delivered via conventional delivery methods (liposomes, polycationic amino polymers, or vesicles) for therapeutic purposes. (See, e.g., Harrington, J. J. et al. (1997) Nat. Genet. 15:345-355.)

For long term production of recombinant proteins in mammalian systems, stable expression of HPEP in cell lines is preferred. For example, sequences encoding HPEP can be transformed into cell lines using expression vectors which may contain viral origins of replication and/or endogenous expression elements and a selectable marker gene on the same or on a separate vector. Following the introduction of the vector, cells may be allowed to grow for about 1 to 2 days in enriched media before being switched to selective media. The purpose of the selectable marker is to confer resistance to a selective agent, and its presence allows growth and recovery of cells which successfully express the introduced sequences. Resistant clones of stably transformed cells may be propagated using tissue culture techniques appropriate to the cell type.

Any number of selection systems may be used to recover transformed cell lines. These include, but are not limited to, the herpes simplex virus thymidine kinase and adenine phosphoribosyltransferase genes, for use in tk- and apr- cells, respectively. (See, e.g., Wigler, M. et al. (1977) Cell 11:223-232; Lowy, I. et al. (1980) Cell 22:817-823.) Also, antimetabolite, antibiotic, or herbicide resistance can be used as the basis for selection. For example, dhfr confers resistance to methotrexate; neo confers resistance to the aminoglycosides neomycin and G-418; and als and pat confer resistance to chlorsulfuron and phosphinotricin acetyltransferase, respectively. (See, e.g., Wigler, M. et al. (1980) Proc. Natl. Acad. Sci. USA 77:3567-3570; Colbere-Garapin, F. et al. (1981) J. Mol. Biol. 150:1-14.) Additional selectable genes have been described, e.g., trpB and hisD, which alter cellular requirements for metabolites. (See, e.g., Hartman, S. C. and R. C. Mulligan (1988) Proc. Natl. Acad. Sci. USA 85:8047-8051.) Visible markers, e.g., anthocyanins, green fluorescent proteins (GFP; Clontech), β glucuronidase and its substrate β-glucuronide, or luciferase and its substrate luciferin may be used. These markers can be used not only to identify transformants, but also to quantify the amount of transient or stable protein expression attributable to a specific vector system. (See, e.g., Rhodes, C. A. (1995) Methods Mol. Biol. 55:121-131.)

Although the presence/absence of marker gene expression suggests that the gene of interest is also present, the presence and expression of the gene may need to be confirmed. For example, if the sequence encoding HPEP is inserted within a marker gene sequence, transformed cells containing sequences encoding HPEP can be identified by the absence of marker gene function. Alternatively, a marker gene can be placed in tandem with a sequence encoding HPEP under the control of a single promoter. Expression of the marker gene in response to induction or selection usually indicates expression of the tandem gene as well.

In general, host cells that contain the nucleic acid sequence encoding HPEP and that express HPEP may be identified by a variety of procedures known to those of skill in the art. These procedures include, but are not limited to, DNA-DNA or DNA-RNA hybridizations, PCR amplification, and protein bioassay or immunoassay techniques which include membrane, solution, or chip based technologies for the detection and/or quantification of nucleic acid or protein sequences.

Immunological methods for detecting and measuring the expression of HPEP using either specific polyclonal or monoclonal antibodies are known in the art. Examples of such techniques include enzyme-linked immunosorbent assays (ELISAs), radioimmunoassays (RIAs), and fluorescence activated cell sorting (FACS). A two-site, monoclonal-based immunoassay utilizing monoclonal antibodies reactive to two non-interfering epitopes on HPEP is preferred, but a competitive binding assay may be employed. These and other assays are well known in the art. (See, e.g., Hampton, R. et al. (1990) Serological Methods a Laboratory Manual, APS Press, St. Paul Minn., to Sect. IV; Coligan, J. E. et al. (1997) Current Protocols in Immunology, Greene Pub. Associates and Wiley-Interscience, New York N.Y.; and Pound, J. D. (1998) Immunochemical Protocols, Humana Press, Totowa N.J.)

A wide variety of labels and conjugation techniques are known by those skilled in the art and may be used in various nucleic acid and amino acid assays. Means for producing labeled hybridization or PCR probes for detecting sequences related to polynucleotides encoding HPEP include oligolabeling, nick translation, end-labeling, or PCR amplification using a labeled nucleotide. Alternatively, the sequences encoding HPEP, or any fragments thereof, may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits, such as those provided by Amersham Pharmacia Biotech, Promega (Madison Wis.), and US Biochemical. Suitable reporter molecules or labels which may be used for ease of detection include radionuclides, enzymes, fluorescent, chemiluminescent, or chromogenic agents, as well as substrates, cofactors, inhibitors, magnetic particles, and the like.

Host cells transformed with nucleotide sequences encoding HPEP may be cultured under conditions suitable for the expression and recovery of the protein from cell culture. The protein produced by a transformed cell may be secreted or retained intracellularly depending on the sequence and/or the vector used. As will be understood by those of skill in the art, expression vectors containing polynucleotides which encode HPEP may be designed to contain signal sequences which direct secretion of HPEP through a prokaryotic or eukaryotic cell membrane.

In addition, a host cell strain may be chosen for its ability to modulate expression of the inserted sequences or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” or “pro” form of the protein may also be used to specify protein targeting, folding, and/or activity. Different host cells which have specific cellular machinery and characteristic mechanisms for post-translational activities (e.g., CHO, HeLa, MDCK, HEK293, and WI38) are available from the American Type Culture Collection (ATCC, Manassas Va.) and may be chosen to ensure the correct modification and processing of the foreign protein.

In another embodiment of the invention, natural, modified, or recombinant nucleic acid sequences encoding HPEP may be ligated to a heterologous sequence resulting in translation of a fusion protein in any of the aforementioned host systems. For example, a chimeric HPEP protein containing a heterologous moiety that can be recognized by a commercially available antibody may facilitate the screening of peptide libraries for inhibitors of HPEP activity. Heterologous protein and peptide moieties may also facilitate purification of fusion proteins using commercially available affinity matrices. Such moieties include, but are not limited to, glutathione S-transferase (GST), maltose binding protein (MBP), thioredoxin (Trx), calmodulin binding peptide (CBP), 6-His, FLAG, c-myc, and hemagglutinin (HA). GST, MBP, Trx, CBP, and 6-His enable purification of their cognate fusion proteins on immobilized glutathione, maltose, phenylarsine oxide, calmodulin, and metal-chelate resins, respectively. FLAG, c-myc, and hemagglutinin (HA) enable immunoaffinity purification of fusion proteins using commercially available monoclonal and polyclonal antibodies that specifically recognize these epitope tags. A fusion protein may also be engineered to contain a proteolytic cleavage site located between the HPEP encoding sequence and the heterologous protein sequence, so that HPEP may be cleaved away from the heterologous moiety following purification. Methods for fusion protein expression and purification are discussed in Ausubel (1995, supra, ch. 10). A variety of commercially available kits may also be used to facilitate expression and purification of fusion proteins.

In a further embodiment of the invention, synthesis of radiolabeled HPEP may be achieved in vitro using the TNT rabbit reticulocyte lysate or wheat germ extract system (Promega). These systems couple transcription and translation of protein-coding sequences operably associated with the T7, T3, or SP6 promoters. Translation takes place in the presence of a radiolabeled amino acid precursor, for example, ³⁵S-methionine.

Fragments of HPEP may be produced not only by recombinant means, but also by direct peptide synthesis using solid-phase techniques. (See, e.g., Creighton, supra. pp. 55-60.) Protein synthesis may be performed by manual techniques or by automation. Automated synthesis may be achieved, for example, using the ABI 431A peptide synthesizer (Perkin-Elmer). Various fragments of HPEP may be synthesized separately and then combined to produce the full length molecule.

Therapeutics

Chemical and structural similarity, e.g., in the context of sequences and motifs, exists between regions of HPEP and human peptidases. In addition, the expression of HPEP is closely associated with cancer and cell proliferation, inflammation and immune response, reproductive tissues, hematopoietic/immune tissues, gastrointestinal tissues, and nervous tissues. Therefore, HPEP appears to play a role in cell proliferative, autoimmune/inflammatory, and metabolic disorders. In the treatment of disorders associated with increased HPEP expression or activity, it is desirable to decrease the expression or activity of HPEP. In the treatment of disorders associated with decreased HPEP expression or activity, it is desirable to increase the expression or activity of HPEP.

Therefore, in one embodiment, HPEP or a fragment or derivative thereof may be administered to a subject to treat or prevent a disorder associated with decreased expression or activity of HPEP. Examples of such disorders include, but are not limited to, a cell proliferative disorder such as actinic keratosis, arteriosclerosis, atherosclerosis, bursitis, cirrhosis, hepatitis, mixed connective tissue disease (MCTD), myelofibrosis, paroxysmal nocturnal hemoglobinuria, polycythemia vera, psoriasis, primary thrombocythemia, and cancers including adenocarcinoma, leukemia, lymphoma, melanoma, myeloma, sarcoma, teratocarcinoma, and, in particular, cancers of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus; an autoimmune/inflammatory disorder such as acquired immunodeficiency syndrome (AIDS), Addison's disease, adult respiratory distress syndrome, allergies, ankylosing spondylitis, amyloidosis, anemia, asthma, atherosclerosis, autoimmune hemolytic anemia, autoimmune thyroiditis, autoimmune polyenodocrinopathy-candidiasis-ectodermal dystrophy (APECED), bronchitis, cholecystitis, contact dermatitis, Crohn's disease, atopic dermatitis, dermatomyositis, diabetes mellitus, emphysema, episodic lymphopenia with lymphocytotoxins, erythroblastosis fetalis, erythema nodosum, atrophic gastritis, glomerulonephritis, Goodpasture's syndrome, gout, Graves' disease, Hashimoto's thyroiditis, hypereosinophilia, irritable bowel syndrome, multiple sclerosis, myasthenia gravis, myocardial or pericardial inflammation, osteoarthritis, osteoporosis, pancreatitis, polymyositis, psoriasis, Reiter's syndrome, rheumatoid arthritis, scleroderma, Sjögren's syndrome, systemic anaphylaxis, systemic lupus erythematosus, systemic sclerosis, thrombocytopenic purpura, ulcerative colitis, uveitis, Werner syndrome, complications of cancer, hemodialysis, and extracorporeal circulation, viral, bacterial, fungal, parasitic, protozoal, and helminthic infections, and trauma; and a metabolic disorder such as Addison's disease, cerebrotendinous xanthomatosis, congenital adrenal hyperplasia, coumarin resistance, cystic fibrosis, diabetes, fatty hepatocirrhosis, fructose-1,6-diphosphatase deficiency, galactosemia, goiter, glucagonoma, glycogen storage diseases, hereditary fructose intolerance, hyperadrenalism, hypoadrenalism, hyperparathyroidism, hypoparathyroidism, hypercholesterolemia, hyperthyroidism, hypoglycemia, hypothyroidism, hyperlipidemia, hyperlipemia, lipid myopathies, lipodystrophies, lysosomal storage diseases, mannosidosis, neuraminidase deficiency, obesity, pentosuria phenylketonuria, and pseudovitamin D-deficiency rickets.

In another embodiment, a vector capable of expressing HPEP or a fragment or derivative thereof may be administered to a subject to treat or prevent a disorder associated with decreased expression or activity of HPEP including, but not limited to, those described above.

In a further embodiment, a pharmaceutical composition comprising a substantially purified HPEP in conjunction with a suitable pharmaceutical carrier may be administered to a subject to treat or prevent a disorder associated with decreased expression or activity of HPEP including, but not limited to, those provided above.

In still another embodiment, an agonist which modulates the activity of HPEP may be administered to a subject to treat or prevent a disorder associated with decreased expression or activity of HPEP including, but not limited to, those listed above.

In a further embodiment, an antagonist of HPEP may be administered to a subject to treat or prevent a disorder associated with increased expression or activity of HPEP. Examples of such disorders include, but are not limited to, those cell proliferative, autoimmune/inflammatory, and metabolic disorders described above. In one aspect, an antibody which specifically binds HPEP may be used directly as an antagonist or indirectly as a targeting or delivery mechanism for bringing a pharmaceutical agent to cells or tissues which express HPEP.

In an additional embodiment, a vector expressing the complement of the polynucleotide encoding HPEP may be administered to a subject to treat or prevent a disorder associated with increased expression or activity of HPEP including, but not limited to, those described above.

In other embodiments, any of the proteins, antagonists, antibodies, agonists, complementary sequences, or vectors of the invention may be administered in combination with other appropriate therapeutic agents. Selection of the appropriate agents for use in combination therapy may be made by one of ordinary skill in the art, according to conventional pharmaceutical principles. The combination of therapeutic agents may act synergistically to effect the treatment or prevention of the various disorders described above. Using this approach, one may be able to achieve therapeutic efficacy with lower dosages of each agent, thus reducing the potential for adverse side effects.

An antagonist of HPEP may be produced using methods which are generally known in the art. In particular, purified HPEP may be used to produce antibodies or to screen libraries of pharmaceutical agents to identify those which specifically bind HPEP. Antibodies to HPEP may also be generated using methods that are well known in the art. Such antibodies may include, but are not limited to, polyclonal, monoclonal, chimeric, and single chain antibodies, Fab fragments, and fragments produced by a Fab expression library. Neutralizing antibodies (i.e., those which inhibit dimer formation) are generally preferred for therapeutic use.

For the production of antibodies, various hosts including goats, rabbits, rats, mice, humans, and others may be immunized by injection with HPEP or with any fragment or oligopeptide thereof which has immunogenic properties. Depending on the host species, various adjuvants may be used to increase immunological response. Such adjuvants include, but are not limited to, Freund's, mineral gels such as aluminum hydroxide, and surface active substances such as lysolecithin, pluronic polyols, polyanions, peptides, oil emulsions, KLH, and dinitrophenol. Among adjuvants used in humans, BCG (bacilli Calmette-Guerin) and Corynebacterium parvum are especially preferable.

It is preferred that the oligopeptides, peptides, or fragments used to induce antibodies to HPEP have an amino acid sequence consisting of at least about 5 amino acids, and generally will consist of at least about 10 amino acids. It is also preferable that these oligopeptides, peptides, or fragments are identical to a portion of the amino acid sequence of the natural protein and contain the entire amino acid sequence of a small, naturally occurring molecule. Short stretches of HPEP amino acids may be fused with those of another protein, such as KLH, and antibodies to the chimeric molecule may be produced.

Monoclonal antibodies to HPEP may be prepared using any technique which provides for the production of antibody molecules by continuous cell lines in culture. These include, but are not limited to, the hybridoma technique, the human B-cell hybridoma technique, and the EBV-hybridoma technique. (See, e.g., Kohler, G. et al. (1975) Nature 256:495-497; Kozbor, D. et al. (1985) J. Immunol. Methods 81:31-42; Cote, R. J. et al. (1983) Proc. Natl. Acad. Sci. USA 80:2026-2030; and Cole, S. P. et al. (1984) Mol. Cell Biol. 62:109-120.)

In addition, techniques developed for the production of “chimeric antibodies,” such as the splicing of mouse antibody genes to human antibody genes to obtain a molecule with appropriate antigen specificity and biological activity, can be used. (See, e.g., Morrison, S. L. et al. (1984) Proc. Natl. Acad. Sci. USA 81:6851-6855; Neuberger, M. S. et al. (1984) Nature 312:604-608; and Takeda, S. et al. (1985) Nature 314:452-454.) Alternatively, techniques described for the production of single chain antibodies may be adapted, using methods known in the art, to produce HPEP-specific single chain antibodies. Antibodies with related specificity, but of distinct idiotypic composition, may be generated by chain shuffling from random combinatorial immunoglobulin libraries. (See, e.g., Burton, D. R. (1991) Proc. Natl. Acad. Sci. USA 88:10134-10137.)

Antibodies may also be produced by inducing in vivo production in the lymphocyte population or by screening immunoglobulin libraries or panels of highly specific binding reagents as disclosed in the literature. (See, e.g., Orlandi, R. et al. (1989) Proc. Natl. Acad. Sci. USA 86:3833-3837; Winter, G. et al. (1991) Nature 349:293-299.)

Antibody fragments which contain specific binding sites for HPEP may also be generated. For example, such fragments include, but are not limited to, F(ab′)₂ fragments produced by pepsin digestion of the antibody molecule and Fab fragments generated by reducing the disulfide bridges of the F(ab′)2 fragments. Alternatively, Fab expression libraries may be constructed to allow rapid and easy identification of monoclonal Fab fragments with the desired specificity. (See, e.g., Huse, W. D. et al. (1989) Science 246:1275-1281.)

Various immunoassays may be used for screening to identify antibodies having the desired specificity. Numerous protocols for competitive binding or immunoradiometric assays using either polyclonal or monoclonal antibodies with established specificities are well known in the art. Such immunoassays typically involve the measurement of complex formation between HPEP and its specific antibody. A two-site, monoclonal-based immunoassay utilizing monoclonal antibodies reactive to two non-interfering HPEP epitopes is generally used, but a competitive binding assay may also be employed (Pound, supra).

Various methods such as Scatchard analysis in conjunction with radioimmunoassay techniques may be used to assess the affinity of antibodies for HPEP. Affinity is expressed as an association constant, Kₐ, which is defined as the molar concentration of HPEP-antibody complex divided by the molar concentrations of free antigen and free antibody under equilibrium conditions. The Kₐ determined for a preparation of polyclonal antibodies, which are heterogeneous in their affinities for multiple HPEP epitopes, represents the average affinity, or avidity, of the antibodies for HPEP. The Kₐ determined for a preparation of monoclonal antibodies, which are monospecific for a particular HPEP epitope, represents a true measure of affinity. High-affinity antibody preparations with Kₐ ranging from about 10⁹ to 10¹² L/mole are preferred for use in immunoassays in which the HPEP-antibody complex must withstand rigorous manipulations. Low-affinity antibody preparations with Kₐ ranging from about 10⁶ to 10⁷ L/mole are preferred for use in immunopurification and similar procedures which ultimately require dissociation of HPEP, preferably in active form, from the antibody (Catty, D. (1988) Antibodies, Volume I: A Practical Approach, IRL Press, Washington, D.C.; Liddell, J. E. and Cryer, A. (1991) A Practical Guide to Monoclonal Antibodies, John Wiley & Sons, New York N.Y.).

The titer and avidity of polyclonal antibody preparations may be further evaluated to determine the quality and suitability of such preparations for certain downstream applications. For example, a polyclonal antibody preparation containing at least 1-2 mg specific antibody/ml, preferably 5-10 mg specific antibody/ml, is generally employed in procedures requiring precipitation of HPEP-antibody complexes. Procedures for evaluating antibody specificity, titer, and avidity, and guidelines for antibody quality and usage in various applications, are generally available. (See, e.g., Catty, supra, and Coligan et al. supra.)

In another embodiment of the invention, the polynucleotides encoding HPEP, or any fragment or complement thereof, may be used for therapeutic purposes. In one aspect, the complement of the polynucleotide encoding HPEP may be used in situations in which it would be desirable to block the transcription of the mRNA. In particular, cells may be transformed with sequences complementary to polynucleotides encoding HPEP. Thus, complementary molecules or fragments may be used to modulate HPEP activity, or to achieve regulation of gene function. Such technology is now well known in the art, and sense or antisense oligonucleotides or larger fragments can be designed from various locations along the coding or control regions of sequences encoding HPEP.

Expression vectors derived from retroviruses, adenoviruses, or herpes or vaccinia viruses, or from various bacterial plasmids, may be used for delivery of nucleotide sequences to the targeted organ, tissue, or cell population. Methods which are well known to those skilled in the art can be used to construct vectors to express nucleic acid sequences complementary to the polynucleotides encoding HPEP. (See, e.g., Sambrook, supra; Ausubel, 1995, supra.)

Genes encoding HPEP can be turned off by transforming a cell or tissue with expression vectors which express high levels of a polynucleotide, or fragment thereof, encoding HPEP. Such constructs may be used to introduce untranslatable sense or antisense sequences into a cell. Even in the absence of integration into the DNA, such vectors may continue to transcribe RNA molecules until they are disabled by endogenous nucleases. Transient expression may last for a month or more with a non-replicating vector, and may last even longer if appropriate replication elements are part of the vector system.

As mentioned above, modifications of gene expression can be obtained by designing complementary sequences or antisense molecules (DNA, RNA, or PNA) to the control, 5′, or regulatory regions of the gene encoding HPEP. Oligonucleotides derived from the transcription initiation site, e.g., between about positions −10 and +10 from the start site, may be employed. Similarly, inhibition can be achieved using triple helix base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature. (See, e.g., Gee, J. E. et al. (1994) in Huber, B. E. and B. I. Carr, Molecular and Immunologic Approaches, Futura Publishing, Mt. Kisco N.Y., pp. 163-177.) A complementary sequence or antisense molecule may also be designed to block translation of mRNA by preventing the transcript from binding to ribosomes.

Ribozymes, enzymatic RNA molecules, may also be used to catalyze the specific cleavage of RNA. The mechanism of ribozyme action involves sequence-specific hybridization of the ribozyme molecule to complementary target RNA, followed by endonucleolytic cleavage. For example, engineered hammerhead motif ribozyme molecules may specifically and efficiently catalyze endonucleolytic cleavage of sequences encoding HPEP.

Specific ribozyme cleavage sites within any potential RNA target are initially identified by scanning the target molecule for ribozyme cleavage sites, including the following sequences: GUA, GUU, and GUC. Once identified, short RNA sequences of between 15 and 20 ribonucleotides, corresponding to the region of the target gene containing the cleavage site, may be evaluated for secondary structural features which may render the oligonucleotide inoperable. The suitability of candidate targets may also be evaluated by testing accessibility to hybridization with complementary oligonucleotides using ribonuclease protection assays.

Complementary ribonucleic acid molecules and ribozymes of the invention may be prepared by any method known in the art for the synthesis of nucleic acid molecules. These include techniques for chemically synthesizing oligonucleotides such as solid phase phosphoramidite chemical synthesis. Alternatively, RNA molecules may be generated by in vitro and in vivo transcription of DNA sequences encoding HPEP. Such DNA sequences may be incorporated into a wide variety of vectors with suitable RNA polymerase promoters such as T7 or SP6. Alternatively, these cDNA constructs that synthesize complementary RNA, constitutively or inducibly, can be introduced into cell lines, cells, or tissues.

RNA molecules may be modified to increase intracellular stability and half-life. Possible modifications include, but are not limited to, the addition of flanking sequences at the 5′ and/or 3′ ends of the molecule, or the use of phosphorothioate or 2′ O-methyl rather than phosphodiesterase linkages within the backbone of the molecule. This concept is inherent in the production of PNAs and can be extended in all of these molecules by the inclusion of nontraditional bases such as inosine, queosine, and wybutosine, as well as acetyl-, methyl-, thio-, and similarly modified forms of adenine, cytidine, guanine, thymine, and uridine which are not as easily recognized by endogenous endonucleases.

Many methods for introducing vectors into cells or tissues are available and equally suitable for use in vivo, in vitro, and ex vivo. For ex vivo therapy, vectors may be introduced into stem cells taken from the patient and clonally propagated for autologous transplant back into that same patient. Delivery by transfection, by liposome injections, or by polycationic amino polymers may be achieved using methods which are well known in the art. (See, e.g., Goldman, C. K. et al. (1997) Nat. Biotechnol. 15:462-466.)

Any of the therapeutic methods described above may be applied to any subject in need of such therapy, including, for example, mammals such as humans, dogs, cats, cows, horses, rabbits, and monkeys.

An additional embodiment of the invention relates to the administration of a pharmaceutical or sterile composition, in conjunction with a pharmaceutically acceptable carrier, for any of the therapeutic effects discussed above. Such pharmaceutical compositions may consist of HPEP, antibodies to HPEP, and mimetics, agonists, antagonists, or inhibitors of HPEP. The compositions may be administered alone or in combination with at least one other agent, such as a stabilizing compound, which may be administered in any sterile, biocompatible pharmaceutical carrier including, but not limited to, saline, buffered saline, dextrose, and water. The compositions may be administered to a patient alone, or in combination with other agents, drugs, or hormones.

The pharmaceutical compositions utilized in this invention may be administered by any number of routes including, but not limited to, oral, intravenous, intramuscular, intra-arterial, intramedullary, intrathecal, intraventricular, transdermal, subcutaneous, intraperitoneal, intranasal, enteral, topical, sublingual, or rectal means.

In addition to the active ingredients, these pharmaceutical compositions may contain suitable pharmaceutically-acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Maack Publishing, Easton Pa.).

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

Pharmaceutical preparations for oral use can be obtained through combining active compounds with solid excipient and processing the resultant mixture of granules (optionally, after grinding) to obtain tablets or dragee cores. Suitable auxiliaries can be added, if desired. Suitable excipients include carbohydrate or protein fillers, such as sugars, including lactose, sucrose, mannitol, and sorbitol; starch from corn, wheat, rice, potato, or other plants; cellulose, such as methyl cellulose, hydroxypropylmethyl-cellulose, or sodium carboxymethylcellulose; gums, including arabic and tragacanth; and proteins, such as gelatin and collagen. If desired, disintegrating or solubilizing agents may be added, such as the cross-linked polyvinyl pyrrolidone, agar, and alginic acid or a salt thereof, such as sodium alginate.

Dragee cores may be used in conjunction with suitable coatings, such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, i.e., dosage.

Pharmaceutical preparations which can be used orally include push-fit capsules made of gelatin, as well as soft, sealed capsules made of gelatin and a coating, such as glycerol or sorbitol. Push-fit capsules can contain active ingredients mixed with fillers or binders, such as lactose or starches, lubricants, such as talc or magnesium stearate, and, optionally, stabilizers. In soft capsules, the active compounds may be dissolved or suspended in suitable liquids, such as fatty oils, liquid, or liquid polyethylene glycol with or without stabilizers.

Pharmaceutical formulations suitable for parenteral administration may be formulated in aqueous solutions, preferably in physiologically compatible buffers such as Hanks' solution, Ringer's solution, or physiologically buffered saline. Aqueous injection suspensions may contain substances which increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils, such as sesame oil, or synthetic fatty acid esters, such as ethyl oleate, triglycerides, or liposomes. Non-lipid polycationic amino polymers may also be used for delivery. Optionally, the suspension may also contain suitable stabilizers or agents to increase the solubility of the compounds and allow for the preparation of highly concentrated solutions.

For topical or nasal administration, penetrants appropriate to the particular barrier to be permeated are used in the formulation. Such penetrants are generally known in the art.

The pharmaceutical compositions of the present invention may be manufactured in a manner that is known in the art, e.g., by means of conventional mixing, dissolving, granulating, dragee-making, levigating, emulsifying, encapsulating, entrapping, or lyophilizing processes.

The pharmaceutical composition may be provided as a salt and can be formed with many acids, including but not limited to, hydrochloric, sulfuric, acetic, lactic, tartaric, malic, and succinic acids. Salts tend to be more soluble in aqueous or other protonic solvents than are the corresponding free base forms. In other cases, the preparation may be a lyophilized powder which may contain any or all of the following: 1 mM to 50 mM histidine, 0.1% to 2% sucrose, and 2% to 7% mannitol, at a pH range of 4.5 to 5.5, that is combined with buffer prior to use.

After pharmaceutical compositions have been prepared, they can be placed in an appropriate container and labeled for treatment of an indicated condition. For administration of HPEP, such labeling would include amount, frequency, and method of administration.

Pharmaceutical compositions suitable for use in the invention include compositions wherein the active ingredients are contained in an effective amount to achieve the intended purpose. The determination of an effective dose is well within the capability of those skilled in the art.

For any compound, the therapeutically effective dose can be estimated initially either in cell culture assays, e.g., of neoplastic cells, or in animal models such as mice, rats, rabbits, dogs, or pigs. An animal model may also be used to determine the appropriate concentration range and route of administration. Such information can then be used to determine useful doses and routes for administration in humans.

A therapeutically effective dose refers to that amount of active ingredient, for example HPEP or fragments thereof, antibodies of HPEP, and agonists, antagonists or inhibitors of HPEP, which ameliorates the symptoms or condition. Therapeutic efficacy and toxicity may be determined by standard pharmaceutical procedures in cell cultures or with experimental animals, such as by calculating the ED₅₀ (the dose therapeutically effective in 50% of the population) or LD₅₀ (the dose lethal to 50% of the population) statistics. The dose ratio of toxic to therapeutic effects is the therapeutic index, which can be expressed as the LD₅₀/ED₅₀ ratio. Pharmaceutical compositions which exhibit large therapeutic indices are preferred. The data obtained from cell culture assays and animal studies are used to formulate a range of dosage for human use. The dosage contained in such compositions is preferably within a range of circulating concentrations that includes the ED₅₀ with little or no toxicity. The dosage varies within this range depending upon the dosage form employed, the sensitivity of the patient, and the route of administration.

The exact dosage will be determined by the practitioner, in light of factors related to the subject requiring treatment. Dosage and administration are adjusted to provide sufficient levels of the active moiety or to maintain the desired effect. Factors which may be taken into account include the severity of the disease state, the general health of the subject, the age, weight, and gender of the subject, time and frequency of administration, drug combination(s), reaction sensitivities, and response to therapy. Long-acting pharmaceutical compositions may be administered every 3 to 4 days, every week, or biweekly depending on the half-life and clearance rate of the particular formulation.

Normal dosage amounts may vary from about 0.1 μg to 100,000 μg, up to a total dose of about 1 gram, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc.

Diagnostics

In another embodiment, antibodies which specifically bind HPEP may be used for the diagnosis of disorders characterized by expression of HPEP, or in assays to monitor patients being treated with HPEP or agonists, antagonists, or inhibitors of HPEP. Antibodies useful for diagnostic purposes may be prepared in the same manner as described above for therapeutics. Diagnostic assays for HPEP include methods which utilize the antibody and a label to detect HPEP in human body fluids or in extracts of cells or tissues. The antibodies may be used with or without modification, and may be labeled by covalent or non-covalent attachment of a reporter molecule. A wide variety of reporter molecules, several of which are described above, are known in the art and may be used.

A variety of protocols for measuring HPEP, including ELISAs, RIAs, and FACS, are known in the art and provide a basis for diagnosing altered or abnormal levels of HPEP expression. Normal or standard values for HPEP expression are established by combining body fluids or cell extracts taken from normal mammalian subjects, for example, human subjects, with antibody to HPEP under conditions suitable for complex formation. The amount of standard complex formation may be quantitated by various methods, such as photometric means. Quantities of HPEP expressed in subject, control, and disease samples from biopsied tissues are compared with the standard values. Deviation between standard and subject values establishes the parameters for diagnosing disease.

In another embodiment of the invention, the polynucleotides encoding HPEP may be used for diagnostic purposes. The polynucleotides which may be used include oligonucleotide sequences, complementary RNA and DNA molecules, and PNAs. The polynucleotides may be used to detect and quantify gene expression in biopsied tissues in which expression of HPEP may be correlated with disease. The diagnostic assay may be used to determine absence, presence, and excess expression of HPEP, and to monitor regulation of HPEP levels during therapeutic intervention.

In one aspect, hybridization with PCR probes which are capable of detecting polynucleotide sequences, including genomic sequences, encoding HPEP or closely related molecules may be used to identify nucleic acid sequences which encode HPEP. The specificity of the probe, whether it is made from a highly specific region, e.g., the 5′ regulatory region, or from a less specific region, e.g., a conserved motif, and the stringency of the hybridization or amplification will determine whether the probe identifies only naturally occurring sequences encoding HPEP, allelic variants, or related sequences.

Probes may also be used for the detection of related sequences, and may have at least 50% sequence identity to any of the HPEP encoding sequences. The hybridization probes of the subject invention may be DNA or RNA and may be derived from the sequence of SEQ ID NO:19-36 or from genomic sequences including promoters, enhancers, and introns of the HPEP gene.

Means for producing specific hybridization probes for DNAs encoding HPEP include the cloning of polynucleotide sequences encoding HPEP or HPEP derivatives into vectors for the production of mRNA probes. Such vectors are known in the art, are commercially available, and may be used to synthesize RNA probes in vitro by means of the addition of the appropriate RNA polymerases and the appropriate labeled nucleotides. Hybridization probes may be labeled by a variety of reporter groups, for example, by radionuclides such as ³²P or ³⁵S, or by enzymatic labels, such as alkaline phosphatase coupled to the probe via avidin/biotin coupling systems, and the like.

Polynucleotide sequences encoding HPEP may be used for the diagnosis of disorders associated with expression of HPEP. Examples of such disorders include, but are not limited to, a cell proliferative disorder such as actinic keratosis, arteriosclerosis, atherosclerosis, bursitis, cirrhosis, hepatitis, mixed connective tissue disease (MCTD), myelofibrosis, paroxysmal nocturnal hemoglobinuria, polycythemia vera, psoriasis, primary thrombocythemia, and cancers including adenocarcinoma, leukemia, lymphoma, melanoma, myeloma, sarcoma, teratocarcinoma, and, in particular, cancers of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus; an autoimmune/inflammatory disorder such as acquired immunodeficiency syndrome (AIDS), Addison's disease, adult respiratory distress syndrome, allergies, ankylosing spondylitis, amyloidosis, anemia, asthma, atherosclerosis, autoimmune hemolytic anemia, autoimmune thyroiditis, autoimmune polyenodocrinopathy-candidiasis-ectodermal dystrophy (APECED), bronchitis, cholecystitis, contact dermatitis, Crohn's disease, atopic dermatitis, dermatomyositis, diabetes mellitus, emphysema, episodic lymphopenia with lymphocytotoxins, erythroblastosis fetalis, erythema nodosum, atrophic gastritis, glomerulonephritis, Goodpasture's syndrome, gout, Graves' disease, Hashimoto's thyroiditis, hypereosinophilia, irritable bowel syndrome, multiple sclerosis, myasthenia gravis, myocardial or pericardial inflammation, osteoarthritis, osteoporosis, pancreatitis, polymyositis, psoriasis, Reiter's syndrome, rheumatoid arthritis, scleroderma, Sjögren's syndrome, systemic anaphylaxis, systemic lupus erythematosus, systemic sclerosis, thrombocytopenic purpura, ulcerative colitis, uveitis, Werner syndrome, complications of cancer, hemodialysis, and extracorporeal circulation, viral, bacterial, fungal, parasitic, protozoal, and helminthic infections, and trauma; and a metabolic disorder such as Addison's disease, cerebrotendinous xanthomatosis, congenital adrenal hyperplasia, coumarin resistance, cystic fibrosis, diabetes, fatty hepatocirrhosis, fructose-1,6-diphosphatase deficiency, galactosemia, goiter, glucagonoma, glycogen storage diseases, hereditary fructose intolerance, hyperadrenalism, hypoadrenalism, hyperparathyroidism, hypoparathyroidism, hypercholesterolemia, hyperthyroidism, hypoglycemia, hypothyroidism, hyperlipidemia, hyperlipemia, lipid myopathies, lipodystrophies, lysosomal storage diseases, mannosidosis, neuraminidase deficiency, obesity, pentosuria phenylketonuria, and pseudovitamin D-deficiency rickets. The polynucleotide sequences encoding HPEP may be used in Southern or northern analysis, dot blot, or other membrane-based technologies; in PCR technologies; in dipstick, pin, and multiformat ELISA-like assays; and in microarrays utilizing fluids or tissues from patients to detect altered HPEP expression. Such qualitative or quantitative methods are well known in the art.

In a particular aspect, the nucleotide sequences encoding HPEP may be useful in assays that detect the presence of associated disorders, particularly those mentioned above. The nucleotide sequences encoding HPEP may be labeled by standard methods and added to a fluid or tissue sample from a patient under conditions suitable for the formation of hybridization complexes. After a suitable incubation period, the sample is washed and the signal is quantified and compared with a standard value. If the amount of signal in the patient sample is significantly altered in comparison to a control sample then the presence of altered levels of nucleotide sequences encoding HPEP in the sample indicates the presence of the associated disorder. Such assays may also be used to evaluate the efficacy of a particular therapeutic treatment regimen in animal studies, in clinical trials, or to monitor the treatment of an individual patient.

In order to provide a basis for the diagnosis of a disorder associated with expression of HPEP, a normal or standard profile for expression is established. This may be accomplished by combining body fluids or cell extracts taken from normal subjects, either animal or human, with a sequence, or a fragment thereof, encoding HPEP, under conditions suitable for hybridization or amplification. Standard hybridization may be quantified by comparing the values obtained from normal subjects with values from an experiment in which a known amount of a substantially purified polynucleotide is used. Standard values obtained in this manner may be compared with values obtained from samples from patients who are symptomatic for a disorder. Deviation from standard values is used to establish the presence of a disorder.

Once the presence of a disorder is established and a treatment protocol is initiated, hybridization assays may be repeated on a regular basis to determine if the level of expression in the patient begins to approximate that which is observed in the normal subject. The results obtained from successive assays may be used to show the efficacy of treatment over a period ranging from several days to months.

With respect to cancer, the presence of an abnormal amount of transcript (either under- or overexpressed) in biopsied tissue from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier thereby preventing the development or further progression of the cancer.

Additional diagnostic uses for oligonucleotides designed from the sequences encoding HPEP may involve the use of PCR. These oligomers may be chemically synthesized, generated enzymatically, or produced in vitro. Oligomers will preferably contain a fragment of a polynucleotide encoding HPEP, or a fragment of a polynucleotide complementary to the polynucleotide encoding HPEP, and will be employed under optimized conditions for identification of a specific gene or to condition. Oligomers may also be employed under less stringent conditions for detection or quantification of closely related DNA or RNA sequences.

Methods which may also be used to quantify the expression of HPEP include radiolabeling or biotinylating nucleotides, coamplification of a control nucleic acid, and interpolating results from standard curves. (See, e.g., Melby, P. C. et al. (1993) J. Immunol. Methods 159:235-244; Duplaa, C. et al. (1993) Anal. Biochem. 212:229-236.) The speed of quantitation of multiple samples may be accelerated by running the assay in a high-throughput format where the oligomer of interest is presented in various dilutions and a spectrophotometric or colorimetric response gives rapid quantitation.

In further embodiments, oligonucleotides or longer fragments derived from any of the polynucleotide sequences described herein may be used as targets in a microarray. The microarray can be used to monitor the expression level of large numbers of genes simultaneously and to identify genetic variants, mutations, and polymorphisms. This information may be used to determine gene function, to understand the genetic basis of a disorder, to diagnose a disorder, and to develop and monitor the activities of therapeutic agents.

Microarrays may be prepared, used, and analyzed using methods known in the art. (See, e.g., Brennan, T. M. et al. (1995) U.S. Pat. No. 5,474,796; Schena, M. et al. (1996) Proc. Natl. Acad. Sci. USA 93:10614-10619; Baldeschweiler et al. (1995) PCT application WO95/251116; Shalon, D. et al. (1995) PCT application WO95/35505; Heller, R. A. et al. (1997) Proc. Natl. Acad. Sci. USA 94:2150-2155; and Heller, M. J. et al. (1997) U.S. Pat. No. 5,605,662.)

In another embodiment of the invention, nucleic acid sequences encoding HPEP may be used to generate hybridization probes useful in mapping the naturally occurring genomic sequence. The sequences may be mapped to a particular chromosome, to a specific region of a chromosome, or to artificial chromosome constructions, e.g., human artificial chromosomes (HACs), yeast artificial chromosomes (YACs), bacterial artificial chromosomes (BACs), bacterial P1 constructions, or single chromosome cDNA libraries. (See, e.g., Harrington, J. J. et al. (1997) Nat. Genet. 15:345-355; Price, C. M. (1993) Blood Rev. 7:127-134; and Trask, B. J. (1991) Trends Genet. 7:149-154.)

Fluorescent in situ hybridization (FISH) may be correlated with other physical chromosome mapping techniques and genetic map data. (See, e.g., Heinz-Ulrich, et al. (1995) in Meyers, supra, pp. 965-968.) Examples of genetic map data can be found in various scientific journals or at the Online Mendelian Inheritance in Man (OMIM) World Wide Web site. Correlation between the location of the gene encoding HPEP on a physical chromosomal map and a specific disorder, or a predisposition to a specific disorder, may help define the region of DNA associated with that disorder. The nucleotide sequences of the invention may be used to detect differences in gene sequences among normal, carrier, and affected individuals.

In situ hybridization of chromosomal preparations and physical mapping techniques, such as linkage analysis using established chromosomal markers, may be used for extending genetic maps. Often the placement of a gene on the chromosome of another mammalian species, such as mouse, may reveal associated markers even if the number or arm of a particular human chromosome is not known. New sequences can be assigned to chromosomal arms by physical mapping. This provides valuable information to investigators searching for disease genes using positional cloning or other gene discovery techniques. Once the disease or syndrome has been crudely localized by genetic linkage to a particular genomic region, e.g., ataxia-telangiectasia to 11q22-23, any sequences mapping to that area may represent associated or regulatory genes for further investigation. (See, e.g., Gatti, R. A. et al. (1988) Nature 336:577-580.) The nucleotide sequence of the subject invention may also be used to detect differences in the chromosomal location due to translocation, inversion, etc., among normal, carrier, or affected individuals.

In another embodiment of the invention, HPEP, its catalytic or immunogenic fragments, or oligopeptides thereof can be used for screening libraries of compounds in any of a variety of drug screening techniques. The fragment employed in such screening may be free in solution, affixed to a solid support, borne on a cell surface, or located intracellularly. The formation of binding complexes between HPEP and the agent being tested may be measured.

Another technique for drug screening provides for high throughput screening of compounds having suitable binding affinity to the protein of interest. (See, e.g., Geysen, et al. (1984) PCT application WO84/03564.) In this method, large numbers of different small test compounds are synthesized on a solid substrate. The test compounds are reacted with HPEP, or fragments thereof, and washed. Bound HPEP is then detected by methods well known in the art. Purified HPEP can also be coated directly onto plates for use in the aforementioned drug screening techniques. Alternatively, non-neutralizing antibodies can be used to capture the peptide and immobilize it on a solid support.

In another embodiment, one may use competitive drug screening assays in which neutralizing antibodies capable of binding HPEP specifically compete with a test compound for binding HPEP. In this manner, antibodies can be used to detect the presence of any peptide which shares one or more antigenic determinants with HPEP.

In additional embodiments, the nucleotide sequences which encode HPEP may be used in any molecular biology techniques that have yet to be developed, provided the new techniques rely on properties of nucleotide sequences that are currently known, including, but not limited to, such properties as the triplet genetic code and specific base pair interactions.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

The disclosures of all patents, applications, and publications mentioned above and below, in particular U.S. Ser. No. 60/172,247, U.S. Ser. No. 60/132,253, and U.S. Ser. No. 60/136,653, are hereby expressly incorporated by reference.

### EXAMPLES

I. Construction of cDNA Libraries

RNA was purchased from Clontech or isolated from tissues described in Table 4. Some tissues were homogenized and lysed in guanidinium isothiocyanate, while others were homogenized and lysed in phenol or in a suitable mixture of denaturants, such as TRIZOL (Life Technologies), a monophasic solution of phenol and guanidine isothiocyanate. The resulting lysates were centrifuged over CsCl cushions or extracted with chloroform. RNA was precipitated from the lysates with either isopropanol or sodium acetate and ethanol, or by other routine methods.

Phenol extraction and precipitation of RNA were repeated as necessary to increase RNA purity. In some cases, RNA was treated with DNase. For most libraries, poly(A+) RNA was isolated using oligo d(T)-coupled paramagnetic particles (Promega), OLIGOTEX latex particles (QIAGEN, Chatsworth Calif.), or an OLIGOTEX mRNA purification kit (QIAGEN). Alternatively, RNA was isolated directly from tissue lysates using other RNA isolation kits, e.g., the POLY(A)PURE mRNA purification kit (Ambion, Austin Tex.).

In some cases, Stratagene was provided with RNA and constructed the corresponding cDNA libraries. Otherwise, cDNA was synthesized and cDNA libraries were constructed with the UNIZAP vector system (Stratagene) or SUPERSCRIPT plasmid system (Life Technologies), using the recommended procedures or similar methods known in the art. (See, e.g., Ausubel, 1997, supra, units 5.1-6.6.) Reverse transcription was initiated using oligo d(T) or random primers. Synthetic oligonucleotide adapters were ligated to double stranded cDNA, and the cDNA was digested with the appropriate restriction enzyme or enzymes. For most libraries, the cDNA was size-selected (300-1000 bp) using SEPHACRYL S1000, SEPHAROSE CL2B, or SEPHAROSE CL4B column chromatography (Amersham Pharmacia Biotech) or preparative agarose gel electrophoresis. cDNAs were ligated into compatible restriction enzyme sites of the polylinker of a suitable plasmid, e.g., PBLUESCRIPT plasmid (Stratagene), PSPORT1 plasmid (Life Technologies), or pINCY (Incyte Pharmaceuticals, Palo Alto Calif.). Recombinant plasmids were transformed into competent E. coli cells including XL1-Blue, XL1-BlueMRF, or SOLR from Stratagene or DH5α, DH10B, or ElectroMAX DH10B from Life Technologies.

II. Isolation of cDNA Clones

Plasmids were recovered from host cells by in vivo excision using the UNIZAP vector system (Stratagene) or by cell lysis. Plasmids were purified using at least one of the following: a Magic or WIZARD Minipreps DNA purification system (Promega); an AGTC Miniprep purification kit (Edge Biosystems, Gaithersburg Md.); and QIAWELL 8 Plasmid, QIAWELL 8 Plus Plasmid, QIAWELL 8 Ultra Plasmid purification systems or the R.E.A.L. PREP 96 plasmid purification kit from QIAGEN. Following precipitation, plasmids were resuspended in 0.1 ml of distilled water and stored, with or without lyophilization, at 4° C.

Alternatively, plasmid DNA was amplified from host cell lysates using direct link PCR in a high-throughput format (Rao, V. B. (1994) Anal. Biochem. 216:1-14). Host cell lysis and thermal cycling steps were carried out in a single reaction mixture. Samples were processed and stored in 384-well plates, and the concentration of amplified plasmid DNA was quantified fluorometrically using PICOGREEN dye (Molecular Probes, Eugene Oreg.) and a FLUOROSKAN II fluorescence scanner (Labsystems Oy, Helsinki, Finland).

III. Sequencing and Analysis

cDNA sequencing reactions were processed using standard methods or high-throughput instrumentation such as the ABI CATALYST 800 (Perkin-Elmer) thermal cycler or the PTC-200 thermal cycler (MJ Research) in conjunction with the HYDRA microdispenser (Robbins Scientific) or the MICROLAB 2200 (Hamilton) liquid transfer system. cDNA sequencing reactions were prepared using reagents provided by Amersham Pharmacia Biotech or supplied in ABI sequencing kits such as the ABI PRISM BIGDYE Terminator cycle sequencing ready reaction kit (Perkin-Elmer). Electrophoretic separation of cDNA sequencing reactions and detection of labeled polynucleotides were carried out using the MEGABACE 1000 DNA sequencing system (Molecular Dynamics); the ABI PRISM 373 or 377 sequencing system (Perkin-Elmer) in conjunction with standard ABI protocols and base calling software; or other sequence analysis systems known in the art. Reading frames within the cDNA sequences were identified using standard methods (reviewed in Ausubel, 1997, supra, unit 7.7). Some of the cDNA sequences were selected for extension using the techniques disclosed in Example VI.

The polynucleotide sequences derived from cDNA sequencing were assembled and analyzed using a combination of software programs which utilize algorithms well known to those skilled in the art. Table 5 summarizes the tools, programs, and algorithms used and provides applicable descriptions, references, and threshold parameters. The first column of Table 5 shows the tools, programs, and algorithms used, the second column provides brief descriptions thereof, the third column presents appropriate references, all of which are incorporated by reference herein in their entirety, and the fourth column presents, where applicable, the scores, probability values, and other parameters used to evaluate the strength of a match between two sequences (the higher the score, the greater the homology between two sequences). Sequences were analyzed using MACDNASIS PRO software (Hitachi Software Engineering, South San Francisco Calif.) and LASERGENE software (DNASTAR). Polynucleotide and polypeptide sequence alignments were generated using the default parameters specified by the clustal algorithm as incorporated into the MEGALIGN multisequence alignment program (DNASTAR), which also calculates the percent identity between aligned sequences.

The polynucleotide sequences were validated by removing vector, linker, and polyA sequences and by masking ambiguous bases, using algorithms and programs based on BLAST, dynamic programing, and dinucleotide nearest neighbor analysis. The sequences were then queried against a selection of public databases such as the GenBank primate, rodent, mammalian, vertebrate, and eukaryote databases, and BLOCKS, PRINTS, DOMO, PRODOM, and PFAM to acquire annotation using programs based on BLAST, FASTA, and BLIMPS. The sequences were assembled into full length polynucleotide sequences using programs based on Phred, Phrap, and Consed, and were screened for open reading frames using programs based on GeneMark, BLAST, and FASTA. The full length polynucleotide sequences were translated to derive the corresponding full length amino acid sequences, and these full length sequences were subsequently analyzed by querying against databases such as the GenBank databases (described above), SwissProt, BLOCKS, PRINTS, DOMO, PRODOM, Prosite, and Hidden Markov Model (HMM)-based protein family databases such as PFAM. HMM is a probabilistic approach which analyzes consensus primary structures of gene families. (See, e.g., Eddy, S. R. (1996) Curr. Opin. Struct. Biol. 6:361-365.)

The programs described above for the assembly and analysis of full length polynucleotide and amino acid sequences were also used to identify polynucleotide sequence fragments from SEQ ID NO:19-36. Fragments from about 20 to about 4000 nucleotides which are useful in hybridization and amplification technologies were described in The Invention section above.

IV. Northern Analysis

Northern analysis is a laboratory technique used to detect the presence of a transcript of a gene and involves the hybridization of a labeled nucleotide sequence to a membrane on which RNAs from a particular cell type or tissue have been bound. (See, e.g., Sambrook, supra, ch. 7; Ausubel, 1995, supra, ch. 4 and 16.)

Analogous computer techniques applying BLAST were used to search for identical or related molecules in nucleotide databases such as GenBank or LIFESEQ (Incyte Pharmaceuticals). This analysis is much faster than multiple membrane-based hybridizations. In addition, the sensitivity of the computer search can be modified to determine whether any particular match is categorized as exact or similar. The basis of the search is the product score, which is defined as:

% sequence identity×% maximum BLAST score/100

The product score takes into account both the degree of similarity between two sequences and the length of the sequence match. For example, with a product score of 40, the match will be exact within a 1% to 2% error, and, with a product score of 70, the match will be exact. Similar molecules are usually identified by selecting those which show product scores between 15 and 40, although lower scores may identify related molecules.

The results of northern analyses are reported as a percentage distribution of libraries in which the transcript encoding HPEP occurred. Analysis involved the categorization of cDNA libraries by organ/tissue and disease. The organ/tissue categories included cardiovascular, dermatologic, developmental, endocrine, gastrointestinal, hematopoietic/immune, musculoskeletal, nervous, reproductive, and urologic. The disease/condition categories included cancer, inflammation, trauma, cell proliferation, neurological, and pooled. For each category, the number of libraries expressing the sequence of interest was counted and divided by the total number of libraries across all categories. Percentage values of tissue-specific and disease- or condition-specific expression are reported in Table 3.

V. Chromosomal Mapping of HPEP Encoding Polynucleotides

The cDNA sequences which were used to assemble SEQ ID NO:30-36 were compared with sequences from the Incyte LIFESEQ database and public domain databases using BLAST and other implementations of the Smith-Waterman algorithm. Sequences from these databases that matched SEQ ID NO:30-36 were assembled into clusters of contiguous and overlapping sequences using assembly algorithms such as Phrap (Table 5). Radiation hybrid and genetic mapping data available from public resources such as the Stanford Human Genome Center (SHGC), Whitehead Institute for Genome Research (WIGR), and Genethon were used to determine if any of the clustered sequences had been previously mapped. Inclusion of a mapped sequence in a cluster resulted in the assignment of all sequences of that cluster, including its particular SEQ ID NO:, to that map location.

The genetic map locations of SEQ ID NO:30 and SEQ ID NO:32 are described in The Invention as ranges, or intervals, of human chromosomes. The map position of an interval, in centiMorgans, is measured relative to the terminus of the chromosome's p-arm. (The centiMorgan (cM) is a unit of measurement based on recombination frequencies between chromosomal markers. On average, 1 cM is roughly equivalent to 1 megabase (Mb) of DNA in humans, although this can vary widely due to hot and cold spots of recombination.) The cM distances are based on genetic markers mapped by Genethon which provide boundaries for radiation hybrid markers whose sequences were included in each of the clusters. Human genome maps and other resources available to the public, such as the NCBI “GeneMap'99” World Wide Web site (ncbi.nlm.nih.gov/genemap/), can be employed to determine if previously identified disease genes map within or in proximity to the intervals indicated above.

VI. Extension of HPEP Encoding Polynucleotides

The full length nucleic acid sequences of SEQ ID NO:19-36 were produced by extension of an appropriate fragment of the full length molecule using oligonucleotide primers designed from this fragment. One primer was synthesized to initiate 5′ extension of the known fragment, and the other primer, to initiate 3′ extension of the known fragment. The initial primers were designed using OLIGO 4.06 software (National Biosciences), or another appropriate program, to be about 22 to 30 nucleotides in length, to have a GC content of about 50% or more, and to anneal to the target sequence at temperatures of about 68° C. to about 72° C. Any stretch of nucleotides which would result in hairpin structures and primer-primer dimerizations was avoided.

Selected human cDNA libraries were used to extend the sequence. If more than one extension was necessary or desired, additional or nested sets of primers were designed.

High fidelity amplification was obtained by PCR using methods well known in the art. PCR was performed in 96-well plates using the PTC-200 thermal cycler (MJ Research, Inc.). The reaction mix contained DNA template, 200 nmol of each primer, reaction buffer containing Mg²⁺, (NH₄)₂SO₄, and β-mercaptoethanol, Taq DNA polymerase (Amersham Pharmacia Biotech), ELONGASE enzyme (Life Technologies), and Pfu DNA polymerase (Stratagene), with the following parameters for primer pair PCI A and PCI B: Step 1: 94° C., 3 min; Step 2: 94° C., 15 sec; Step 3: 60° C., 1 min; Step 4: 68° C., 2 min; Step 5: Steps 2, 3, and 4 repeated 20 times; Step 6: 68° C., 5 min; Step 7: storage at 4° C. In the alternative, the parameters for primer pair T7 and SK+ were as follows: Step 1: 94° C., 3 min; Step 2: 94° C., 15 sec; Step 3: 57° C., 1 min; Step 4: 68° C., 2 min; Step 5: Steps 2, 3, and 4 repeated 20 times; Step 6: 68° C., 5 min; Step 7: storage at 4° C.

The concentration of DNA in each well was determined by dispensing 100 μl PICOGREEN quantitation reagent (0.25% (v/v) PICOGREEN; Molecular Probes, Eugene Oreg.) dissolved in 1× TE and 0.5 μl of undiluted PCR product into each well of an opaque fluorimeter plate (Corning Costar, Acton Mass.), allowing the DNA to bind to the reagent. The plate was scanned in a Fluoroskan II (Labsystems Oy, Helsinki, Finland) to measure the fluorescence of the sample and to quantify the concentration of DNA. A 5 μl to 10 μl aliquot of the reaction mixture was analyzed by electrophoresis on a 1% agarose mini-gel to determine which reactions were successful in extending the sequence.

The extended nucleotides were desalted and concentrated, transferred to 384-well plates, digested with CviJI cholera virus endonuclease (Molecular Biology Research, Madison Wis.), and sonicated or sheared prior to religation into pUC 18 vector (Amersham Pharmacia Biotech). For shotgun sequencing, the digested nucleotides were separated on low concentration (0.6 to 0.8%) agarose gels, fragments were excised, and agar digested with Agar ACE (Promega). Extended clones were religated using T4 ligase (New England Biolabs, Beverly Mass.) into pUC 18 vector (Amersham Pharmacia Biotech), treated with Pfu DNA polymerase (Stratagene) to fill-in restriction site overhangs, and transfected into competent E. coli cells. Transformed cells were selected on antibiotic-containing media, individual colonies were picked and cultured overnight at 37° C. in 384-well plates in LB/2× carb liquid media.

The cells were lysed, and DNA was amplified by PCR using Taq DNA polymerase (Amersham Pharmacia Biotech) and Pfu DNA polymerase (Stratagene) with the following parameters: Step 1: 94° C., 3 min; Step 2: 94° C., 15 sec; Step 3: 60° C., 1 min; Step 4: 72° C., 2 min; Step 5: steps 2, 3, and 4 repeated 29 times; Step 6: 72° C., 5 min; Step 7: storage at 4° C. DNA was quantified by PICOGREEN reagent (Molecular Probes) as described above. Samples with low DNA recoveries were reamplified using the same conditions as described above. Samples were diluted with 20% dimethysulfoxide (1:2, v/v), and sequenced using DYENAMIC energy transfer sequencing primers and the DYENAMIC DIRECT kit (Amersham Pharmacia Biotech) or the ABI PRISM BIGDYE Terminator cycle sequencing ready reaction kit (Perkin-Elmer).

In like manner, the nucleotide sequences of SEQ ID NO:19-36 are used to obtain 5′ regulatory sequences using the procedure above, oligonucleotides designed for such extension, and an appropriate genomic library.

VII. Labeling and Use of Individual Hybridization Probes

Hybridization probes derived from SEQ ID NO:19-36 are employed to screen cDNAs, genomic DNAs, or mRNAs. Although the labeling of oligonucleotides, consisting of about 20 base pairs, is specifically described, essentially the same procedure is used with larger nucleotide fragments. Oligonucleotides are designed using state-of-the-art software such as OLIGO 4.06 software (National Biosciences) and labeled by combining 50 pmol of each oligomer, 250 μCi of [γ-³²P] adenosine triphosphate (Amersham Pharmacia Biotech), and T4 polynucleotide kinase (DuPont NEN, Boston Mass.). The labeled oligonucleotides are substantially purified using a SEPHADEX G-25 superfine size exclusion dextran bead column (Amersham Pharmacia Biotech). An aliquot containing 10⁷ counts per minute of the labeled probe is used in a typical membrane-based hybridization analysis of human genomic DNA digested with one of the following endonucleases: Ase I, Bgl II, Eco RI, Pst I, Xba I, or Pvu II (DuPont NEN).

The DNA from each digest is fractionated on a 0.7% agarose gel and transferred to nylon membranes (Nytran Plus, Schleicher & Schuell, Durham N.H.). Hybridization is carried out for 16 hours at 40° C. To remove nonspecific signals, blots are sequentially washed at room temperature under conditions of up to, for example, 0.1× saline sodium citrate and 0.5% sodium dodecyl sulfate. Hybridization patterns are visualized using autoradiography or an alternative imaging means and compared.

VIII. Microarrays

A chemical coupling procedure and an ink jet device can be used to synthesize array elements on the surface of a substrate. (See, e.g., Baldeschweiler, supra.) An array analogous to a dot or slot blot may also be used to arrange and link elements to the surface of a substrate using thermal, UV, chemical, or mechanical bonding procedures. A typical array may be produced by hand or using available methods and machines and contain any appropriate number of elements. After hybridization, nonhybridized probes are removed and a scanner used to determine the levels and patterns of fluorescence. The degree of complementarity and the relative abundance of each probe which hybridizes to an element on the microarray may be assessed through analysis of the scanned images.

Full-length cDNAs, Expressed Sequence Tags (ESTs), or fragments thereof may comprise the elements of the microarray. Fragments suitable for hybridization can be selected using software well known in the art such as LASERGENE software (DNASTAR). Full-length cDNAs, ESTs, or fragments thereof corresponding to one of the nucleotide sequences of the present invention, or selected at random from a cDNA library relevant to the present invention, are arranged on an appropriate substrate, e.g., a glass slide. The cDNA is fixed to the slide using, e.g., UV cross-linking followed by thermal and chemical treatments and subsequent drying. (See, e.g., Schena, M. et al. (1995) Science 270:467-470; Shalon, D. et al. (1996) Genome Res. 6:639-645.) Fluorescent probes are prepared and used for hybridization to the elements on the substrate. The substrate is analyzed by procedures described above.

IX. Complementary Polynucleotides

Sequences complementary to the HPEP-encoding sequences, or any parts thereof, are used to detect, decrease, or inhibit expression of naturally occurring HPEP. Although use of oligonucleotides comprising from about 15 to 30 base pairs is described, essentially the same procedure is used with smaller or with larger sequence fragments. Appropriate oligonucleotides are designed using OLIGO 4.06 software (National Biosciences) and the coding sequence of HPEP. To inhibit transcription, a complementary oligonucleotide is designed from the most unique 5′ sequence and used to prevent promoter binding to the coding sequence. To inhibit translation, a complementary oligonucleotide is designed to prevent ribosomal binding to the HPEP-encoding transcript.

X. Expression of HPEP

Expression and purification of HPEP is achieved using bacterial or virus-based expression systems. For expression of HPEP in bacteria, cDNA is subcloned into an appropriate vector containing an antibiotic resistance gene and an inducible promoter that directs high levels of cDNA transcription. Examples of such promoters include, but are not limited to, the trp-lac (tac) hybrid promoter and the T5 or T7 bacteriophage promoter in conjunction with the lac operator regulatory element. Recombinant vectors are transformed into suitable bacterial hosts, e.g., BL21(DE3). Antibiotic resistant bacteria express HPEP upon induction with isopropyl beta-D-thiogalactopyranoside (IPTG). Expression of HPEP in eukaryotic cells is achieved by infecting insect or mammalian cell lines with recombinant Autographica californica nuclear polyhedrosis virus (AcMNPV), commonly known as baculovirus. The nonessential polyhedrin gene of baculovirus is replaced with cDNA encoding HPEP by either homologous recombination or bacterial-mediated transposition involving transfer plasmid intermediates. Viral infectivity is maintained and the strong polyhedrin promoter drives high levels of cDNA transcription. Recombinant baculovirus is used to infect Spodoptera frugiperda (Sf9) insect cells in most cases, or human hepatocytes, in some cases. Infection of the latter requires additional genetic modifications to baculovirus. (See Engelhard, E. K. et al. (1994) Proc. Natl. Acad. Sci. USA 91:3224-3227; Sandig, V. et al. (1996) Hum. Gene Ther. 7:1937-1945.)

In most expression systems, HPEP is synthesized as a fusion protein with, e.g., glutathione S-transferase (GST) or a peptide epitope tag, such as FLAG or 6-His, permitting rapid, single-step, affinity-based purification of recombinant fusion protein from crude cell lysates. GST, a 26-kilodalton enzyme from Schistosoma iaponicum, enables the purification of fusion proteins on immobilized glutathione under conditions that maintain protein activity and antigenicity (Amersham Pharmacia Biotech). Following purification, the GST moiety can be proteolytically cleaved from HPEP at specifically engineered sites. FLAG, an 8-amino acid peptide, enables immunoaffinity purification using commercially available monoclonal and polyclonal anti-FLAG antibodies (Eastman Kodak). 6-His, a stretch of six consecutive histidine residues, enables purification on metal-chelate resins (QIAGEN). Methods for protein expression and purification are discussed in Ausubel (1995, supra, ch. 10 and 16). Purified HPEP obtained by these methods can be used directly in the following activity assay.

XI. Demonstration of HPEP Activity

Peptidase activity of HPEP is measured by the hydrolysis of appropriate synthetic peptide substrates conjugated with various chromogenic molecules in which the degree of hydrolysis is quantified by spectrophotometric or fluorometric absorption of the released chromophore (Beynon, R. J. and J. S. Bond (1994) Proteolytic Enzymes: A Practical Approach, Oxford University Press, New York N.Y., pp.25-55). Peptide substrates are designed according to the category of protease activity as endopeptidase (serine, cysteine, aspartic proteases), aminopeptidase (leucine aminopeptidase), or carboxypeptidase (Carboxypeptidase A and B, procollagen C-proteinase). Chromogens commonly used are 2-naphthylamine, 4-nitroaniline, and furylacrylic acid. Assays are performed at room temperature and contain an aliquot of the enzyme and the appropriate substrate in a suitable buffer. Reactions are carried out in an optical cuvette and monitored by measurement of the increase/decrease in absorbance of the chromogen released during hydrolysis of the peptide substrate. The change in absorbance is proportional to the peptidase activity of HPEP in the assay.

Alternatively, regulation of peptidase activity (agonism or antagonism) by HPEP is measured using an appropriate protease assay as described above in the presence or absence of HPEP as an agonist or inhibitor of this activity. Protease activity is measured in the absence of HPEP (control activity) and in the presence of varying amounts of HPEP. The change in protease activity compared to the control is proportional to the amount of HPEP in the assay and is a measure of the protease regulatory activity of HPEP.

Alternatively, ubiquitin activity of HPEP is demonstrated by its ability to form a covalent thiolester bond with ubiquitin-activating enzyme (E1). This activity can be detected and quantified using a “covalent affinity” chromatography procedure (Ciechanover, A. et al. (1982) J. Biol. Chem. 257:2537-2542). El is first conjugated to SEPHAROSE resin, an inert resin, using methods well known by those skilled in the art. HPEP, produced by recombinant methods or purified biochemically, is present in a solution containing ATP and magnesium ions. This solution is exposed to the E1-Sepharose conjugate in a column chromatography format. E1-Sepharose is washed with a solution containing a high concentration of salt, such as sodium chloride. This treatment is effective in removing virtually all proteins that are not covalently bound to E1-Sepharose. HPEP covalently bound to E1-Sepharose is eluted with a thiol compound such as dithiothreitol. The presence of HPEP in the eluent is detected by SDS-polyacrylamide gel electrophoresis and gel staining. Immunological methods such as western blot which utilize specific antibody directed against HPEP are used to quantify the amount of HPEP in the eluent. The amount of HPEP that binds to E1-Sepharose is proportional to the ubiquitin activity of HPEP.

XII. Functional Assays

HPEP function is assessed by expressing the sequences encoding HPEP at physiologically elevated levels in mammalian cell culture systems. cDNA is subcloned into a mammalian expression vector containing a strong promoter that drives high levels of cDNA expression. Vectors of choice include pCMV SPORT (Life Technologies) and pCR3.1 (Invitrogen, Carlsbad Calif.), both of which contain the cytomegalovirus promoter. 5-10 μg of recombinant vector are transiently transfected into a human cell line, for example, an endothelial or hematopoietic cell line, using either liposome formulations or electroporation. 1-2 μg of an additional plasmid containing sequences encoding a marker protein are co-transfected. Expression of a marker protein provides a means to distinguish transfected cells from nontransfected cells and is a reliable predictor of cDNA expression from the recombinant vector. Marker proteins of choice include, e.g., Green Fluorescent Protein (GFP; Clontech), CD64, or a CD64-GFP fusion protein. Flow cytometry (FCM), an automated, laser optics-based technique, is used to identify transfected cells expressing GFP or CD64-GFP and to evaluate the apoptotic state of the cells and other cellular properties. FCM detects and quantifies the uptake of fluorescent molecules that diagnose events preceding or coincident with cell death. These events include changes in nuclear DNA content as measured by staining of DNA with propidium iodide; changes in cell size and granularity as measured by forward light scatter and 90 degree side light scatter; down-regulation of DNA synthesis as measured by decrease in bromodeoxyuridine uptake; alterations in expression of cell surface and intracellular proteins as measured by reactivity with specific antibodies; and alterations in plasma membrane composition as measured by the binding of fluorescein-conjugated Annexin V protein to the cell surface. Methods in flow cytometry are discussed in Ormerod, M. G. (1994) Flow Cytometry, Oxford, New York N.Y.

The influence of HPEP on gene expression can be assessed using highly purified populations of cells transfected with sequences encoding HPEP and either CD64 or CD64-GFP. CD64 and CD64-GFP are expressed on the surface of transfected cells and bind to conserved regions of human immunoglobulin G (IgG). Transfected cells are efficiently separated from nontransfected cells using magnetic beads coated with either human IgG or antibody against CD64 (DYNAL, Lake Success N.Y.). mRNA can be purified from the cells using methods well known by those of skill in the art. Expression of mRNA encoding HPEP and other genes of interest can be analyzed by northern analysis or microarray techniques.

XIII. Production of HPEP Specific Antibodies

HPEP substantially purified using polyacrylamide gel electrophoresis (PAGE; see, e.g., Harrington, M. G. (1990) Methods Enzymol. 182:488-495), or other purification techniques, is used to immunize rabbits and to produce antibodies using standard protocols.

Alternatively, the HPEP amino acid sequence is analyzed using LASERGENE software (DNASTAR) to determine regions of high immunogenicity, and a corresponding oligopeptide is synthesized and used to raise antibodies by means known to those of skill in the art. Methods for selection of appropriate epitopes, such as those near the C-terminus or in hydrophilic regions are well described in the art. (See, e.g., Ausubel, 1995, supra, ch. 11.)

Typically, oligopeptides of about 15 residues in length are synthesized using an ABI 431A peptide synthesizer (Perkin-Elmer) using fmoc-chemistry and coupled to KLH (Sigma-Aldrich, St. Louis Mo.) by reaction with N-maleimidobenzoyl-N-hydroxysuccinimide ester (MBS) to increase immunogenicity. (See, e.g., Ausubel, 1995, supra.) Rabbits are immunized with the oligopeptide-KLH complex in complete Freund's adjuvant. Resulting antisera are tested for antipeptide and anti-HPEP activity by, for example, binding the peptide or HPEP to a substrate, blocking with 1% BSA, reacting with rabbit antisera, washing, and reacting with radio-iodinated goat anti-rabbit IgG.

XIV. Purification of Naturally Occurring HPEP Using Specific Antibodies

Naturally occurring or recombinant HPEP is substantially purified by immunoaffinity chromatography using antibodies specific for HPEP. An immunoaffinity column is constructed by covalently coupling anti-HPEP antibody to an activated chromatographic resin, such as CNBr-activated SEPHAROSE (Amersham Pharmacia Biotech). After the coupling, the resin is blocked and washed according to the manufacturer's instructions.

Media containing HPEP are passed over the immunoaffinity column, and the column is washed under conditions that allow the preferential absorbance of HPEP (e.g., high ionic strength buffers in the presence of detergent). The column is eluted under conditions that disrupt antibody/HPEP binding (e.g., a buffer of pH 2 to pH 3, or a high concentration of a chaotrope, such as urea or thiocyanate ion), and HPEP is collected.

XV. Identification of Molecules Which Interact with HPEP

HPEP, or biologically active fragments thereof, are labeled with ¹²⁵I Bolton-Hunter reagent. (See, e.g., Bolton A. E. and W. M. Hunter (1973) Biochem. J. 133:529-539.) Candidate molecules previously arrayed in the wells of a multi-well plate are incubated with the labeled HPEP, washed, and any wells with labeled HPEP complex are assayed. Data obtained using different concentrations of HPEP are used to calculate values for the number, affinity, and association of HPEP with the candidate molecules.

Various modifications and variations of the described methods and systems of the invention will be apparent to those skilled in the art without departing from the scope and spirit of the invention. Although the invention has been described in connection with certain embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

What is claimed is:

1. An isolated polypeptide selected from the group consisting of:
a) a polypeptide comprising an amino acid sequence selected from the group consisting of SEQ ID NO:1-18,
b) a polypeptide comprising a naturally occurring an amino acid sequence at least 90% identical to an amino acid sequence selected from the group consisting of SEQ ID NO:1-18,
c) a biologically active fragment of a polypeptide having an amino acid sequence selected from the group consisting of SEQ ID NO:1-18, and
d) an immunogenic fragment of a polypeptide having an amino acid sequence selected from the group consisting of SEQ ID NO:1-18.

2. An isolated polypeptide of claim 1, comprising an amino acid sequence selected from the group consisting of SEQ ID NO:1-18.

3. An isolated polynucleotide encoding a polypeptide of claim 1.

4. An isolated polynucleotide encoding a polypeptide of claim 2.

5. An isolated polynucleotide of claim 4, having a sequence selected from the group consisting of SEQ ID NO:19-36.

6. A recombinant polynucleotide comprising a promoter sequence operably linked to a polynucleotide of claim 3.

7. A cell transformed with a recombinant polynucleotide of claim 6.

8. A transgenic organism comprising a recombinant polynucleotide of claim 6.

9. A method of producing a polypeptide of claim 1, the method comprising:
a) culturing a cell under conditions suitable for expression of the polypeptide, wherein said cell is transformed with a recombinant polynucleotide, and said recombinant polynucleotide comprises a promoter sequence operably linked to a polynucleotide encoding the polypeptide of claim 1, and
b) recovering the polypeptide so expressed.

10. A method of claim 9, wherein the polypeptide comprises an amino acid sequence selected from the group consisting of SEQ ID NO:1-18.

11. An isolated antibody which specifically binds to a polypeptide of claim 1.

12. An isolated polynucleotide selected from the group consisting of:
a) a polynucleotide comprising a polynucleotide sequence selected from the group consisting of SEQ ID NO:19-36,
b) a polynucleotide comprising a naturally occurring polynucleotide sequence at least 90% identical to a polynucleotide sequence selected from the group consisting of SEQ ID NO:19-36,
c) a polynucleotide complementary to a polynucleotide of a),
d) a polynucleotide complementary to a polynucleotide of b) and
e) an RNA equivalent of a)-d).

13. An isolated polynucleotide comprising at least 60 contiguous nucleotides of a polynucleotide of claim 12.

14. A method of detecting a target polynucleotide in a sample, said target polynucleotide having a sequence of a polynucleotide of claim 12, the method comprising:
a) hybridizing the sample with a probe comprising at least 20 contiguous nucleotides comprising a sequence complementary to said target polynucleotide in the sample, and which probe specifically hybridizes to said target polynucleotide, under conditions whereby a hybridization complex is formed between said probe and said target polynucleotide or fragments thereof, and
b) detecting the presence or absence of said hybridization complex, and, optionally, if present, the amount thereof.

15. A method of claim 14, wherein the probe comprises at least 60 contiguous nucleotides.

16. A method of detecting a target polynucleotide in a sample, said target polynucleotide having a sequence of a polynucleotide of claim 12, the method comprising:
a) amplifying said target polynucleotide or fragment thereof using polymerase chain reaction amplification, and
b) detecting the presence or absence of said amplified target polynucleotide or fragment thereof, and, optionally, if present, the amount thereof.

17. A composition comprising a polypeptide of claim 1 and a pharmaceutically acceptable excipient.

18. A composition of claim 17, wherein the polypeptide comprises an amino acid sequence selected from the group consisting of SEQ ID NO:1-18.

19. A method for treating a disease or condition associated with decreased expression of functional HPEP, comprising administering to a patient in need of such treatment the composition of claim 17.

20. A method of screening a compound for effectiveness as an agonist of a polypeptide of claim 1, the method comprising:
a) contacting a sample comprising a polypeptide of claim 1 with a compound, and
b) detecting agonist activity in the sample.

21. A composition comprising an agonist compound identified by a method of claim 20 and a pharmaceutically acceptable excipient.

22. A method for treating a disease or condition associated with decreased expression of functional HPEP, comprising administering to a patient in need of such treatment a composition of claim 21.

23. A method of screening a compound for effectiveness as an antagonist of a polypeptide of claim 1, the method comprising:
a) contacting a sample comprising a polypeptide of claim 1 with a compound, and
b) detecting antagonist activity in the sample.

24. A composition comprising an antagonist compound identified by a method of claim 23 and a pharmaceutically acceptable excipient.

25. A method for treating a disease or condition associated with overexpression of functional HPEP, comprising administering to a patient in need of such treatment a composition of claim 24.

26. A method of screening for a compound that specifically binds to the polypeptide of claim 1, the method comprising:
a) combining the polypeptide of claim 1 with at least one test compound under suitable conditions, and
b) detecting binding of the polypeptide of claim 1 to the test compound, thereby identifying a compound that specifically binds to the polypeptide of claim 1.

27. A method of screening for a compound that modulates the activity of the polypeptide of claim 1, said method comprising:
a) combining the polypeptide of claim 1 with at least one test compound under conditions permissive for the activity of the polypeptide of claim 1,
b) assessing the activity of the polypeptide of claim 1 in the presence of the test compound, and
c) comparing the activity of the polypeptide of claim 1 in the presence of the test compound with the activity of the polypeptide of claim 1 in the absence of the test compound, wherein a change in the activity of the polypeptide of claim 1 in the presence of the test compound is indicative of a compound that modulates the activity of the polypeptide of claim 1.

28. A method of screening a compound for effectiveness in altering expression of a target polynucleotide, wherein said target polynucleotide comprises a polynucleotide sequence of claim 5, the method comprising:
a) contacting a sample comprising the target polynucleotide with, under conditions suitable for the expression of the target polynucleotide,
b) detecting altered expression of the target polynucleotide, and
c) comparing the expression of the target polynucleotide in the presence of varying amounts of the compound and in the absence of the compound.

29. A method of screening for potential toxicity of a test compound, the method comprising:
a) treating a biological sample containing nucleic acids with the test compound,
b) hybridizing the nucleic acids of the treated biological sample with a probe comprising at least 20 contiguous nucleotides of a polynucleotide of claim 12 under conditions whereby a specific hybridization complex is formed between said probe and a target polynucleotide in the biological sample, said target polynucleotide comprising a polynucleotide sequence of a polynucleotide of claim 12 or fragment thereof,
c) quantifying the amount of hybridization complex, and
d) comparing the amount of hybridization complex in the treated biological sample with the amount of hybridization complex in an untreated biological sample, wherein a difference in the amount of hybridization complex in the treated biological sample indicates potential toxicity of the test compound.

30. A diagnostic test for a condition or disease associated with the expression of HPEP in a biological sample, the method comprising:
a) combining the biological sample with an antibody of claim 11, under conditions suitable for the antibody to bind the polypeptide and form an antibody:polypeptide complex, and
b) detecting the complex, wherein the presence of the complex correlates with the presence of the polypeptide in the biological sample.

31. The antibody of claim 11, wherein the antibody is:
a) a chimeric antibody,
b) a single chain antibody,
c) a Fab fragment,
d) a F(ab′)₂ fragment, or
e) a humanized antibody.

32. A composition comprising an antibody of claim 11 and an acceptable excipient.

33. A method of diagnosing a condition or disease associated with the expression of HPEP in a subject, comprising administering to said subject an effective amount of the composition of claim 32.

34. A composition of claim 32, further comprising a label.

35. A method of diagnosing a condition or disease associated with the expression of HPEP in a subject, comprising administering to said subject an effective amount of the composition of claim 34.

36. A method of preparing a polyclonal antibody with the specificity of the antibody of claim 11, the method comprising:
a) immunizing an animal with a polypeptide consisting of an amino acid sequence selected from the group consisting of SEQ ID NO:1-18, or an immunogenic fragment thereof, under conditions to elicit an antibody response,
b) isolating antibodies from said animal, and
c) screening the isolated antibodies with the polypeptide, thereby identifying a polyclonal antibody which binds specifically to a polypeptide comprising an amino acid sequence selected from the group consisting of SEQ ID NO:1-18.

37. A. polyclonal antibody produced by a method of claim 36.

38. A composition comprising the polyclonal antibody of claim 37 and a suitable carrier.

39. A method of making a monoclonal antibody with the specificity of the antibody of claim 11, the method comprising:
a) immunizing an animal with a polypeptide consisting of an amino acid sequence selected from the group consisting of SEQ ID NO:1-18, or an immunogenic fragment thereof, under conditions to elicit an antibody response,
b) isolating antibody producing cells from the animal,
c) fusing the antibody producing cells with immortalized cells to form monoclonal antibody-producing hybridoma cells,
d) culturing the hybridoma cells, and
e) isolating from the culture monoclonal antibody which binds specifically to a polypeptide comprising an amino acid sequence selected from the group consisting of SEQ ID NO:1-18.

40. A monoclonal antibody produced by a method of claim 39.

41. A composition comprising the monoclonal antibody of claim 40 and a suitable carrier.

42. The antibody of claim 11, wherein the monoclonal antibody is produced by screening a Fab expression library.

43. The antibody of claim 11, wherein the antibody is produced by screening a recombinant immunoglobulin library.

44. A method of detecting a polypeptide comprising an amino acid sequence selected from the group consisting of SEQ ID NO:1-18 in a sample, the method comprising:
a) incubating the antibody of claim 11 with a sample under conditions to allow specific binding of the antibody and the polypeptide, and
b) detecting specific binding, wherein specific binding indicates the presence of a polypeptide comprising an amino acid sequence selected from the group consisting of SEQ ID NO:1-18 in the sample.

45. A method of purifying a polypeptide comprising an amino acid sequence selected from the group consisting of SEQ ID NO:1-18 from a sample, the method comprising:
a) incubating the antibody of claim 11 with a sample under conditions to allow specific binding of the antibody and the polypeptide, and
b) separating the antibody from the sample and obtaining the purified polypeptide comprising an amino acid sequence selected from the group consisting of SEQ ID NO:1-18.

46. A microarray wherein at least one element of the microarray is a polynucleotide of claim 13.

47. A method of generating an expression profile of a sample which contains polynucleotides, the method comprising:
a) labeling the polynucleotides of the sample,
b) contacting the elements of the microarray of claim 46 with the labeled polynucleotides of the sample under conditions suitable for the formation of a hybridization complex, and
c) quantifying the expression of the polynucleotides in the sample.

48. An array comprising different nucleotide molecules affixed in distinct physical locations on a solid substrate, wherein at least one of said nucleotide molecules comprises a first oligonucleotide or polynucleotide sequence specifically hybridizable with at least 30 contiguous nucleotides of a target polynucleotide, and wherein said target polynucleotide is a polynucleotide of claim 12.

49. An array of claim 48, wherein said first oligonucleotide or polynucleotide sequence is completely complementary to at least 30 contiguous nucleotides of said target polynucleotide.

50. An array of claim 48, wherein said first oligonucleotide or polynucleotide sequence is completely complementary to at least 60 contiguous nucleotides of said target polynucleotide.

51. An array of claim 48, wherein said first oligonucleotide or polynucleotide sequence is completely complementary to said target polynucleotide.

52. An array of claim 48, which is a microarray.

53. An array of claim 48, further comprising said target polynucleotide hybridized to a nucleotide molecule comprising said first oligonucleotide or polynucleotide sequence.

54. An array of claim 48, wherein a linker joins at least one of said nucleotide molecules to said solid substrate.

55. An array of claim 48, wherein each distinct physical location on the substrate contains multiple nucleotide molecules, and the multiple nucleotide molecules at any single distinct physical location have the same sequence, and each distinct physical location on the substrate contains nucleotide molecules having a sequence which differs from the sequence of nucleotide molecules at another distinct physical location on the substrate.

56. A polypeptide of claim 1, comprising the amino acid sequence of SEQ ID NO:1.

57. A polypeptide of claim 1, comprising the amino acid sequence of SEQ ID NO:2.

58. A polypeptide of claim 1, comprising the amino acid sequence of SEQ ID NO:3.

59. A polypeptide of claim 1, comprising the amino acid sequence of SEQ ID NO:4.

60. A polypeptide of claim 1, comprising the amino acid sequence of SEQ ID NO:5.

61. A polypeptide of claim 1, comprising the amino acid sequence of SEQ ID NO:6.

62. A polypeptide of claim 1, comprising the amino acid sequence of SEQ ID NO:7.

63. A polypeptide of claim 1, comprising the amino acid sequence of SEQ ID NO:8.

64. A polypeptide of claim 1, comprising the amino acid sequence of SEQ ID NO:9.

65. A polypeptide of claim 1, comprising the amino acid sequence of SEQ ID NO:10.

66. A polypeptide of claim 1, comprising the amino acid sequence of SEQ ID NO:11.

67. A polypeptide of claim 1, comprising the amino acid sequence of SEQ ID NO:12.

68. A polypeptide of claim 1, comprising the amino acid sequence of SEQ ID NO:13.

69. A polypeptide of claim 1, comprising the amino acid sequence of SEQ ID NO:14.

70. A polypeptide of claim 1, comprising the amino acid sequence of SEQ ID NO:15.

71. A polypeptide of claim 1, comprising the amino acid sequence of SEQ ID NO:16.

72. A polypeptide of claim 1, comprising the amino acid sequence of SEQ ID NO:17.

73. A polypeptide of claim 1, comprising the amino acid sequence of SEQ ID NO:18.

74. A polynucleotide of claim 12, comprising the polynucleotide sequence of SEQ ID NO:19.

75. A polynucleotide of claim 12, comprising the polynucleotide sequence of SEQ ID NO:20.

76. A polynucleotide of claim 12, comprising the polynucleotide sequence of SEQ ID NO:21.

77. A polynucleotide of claim 12, comprising the polynucleotide sequence of SEQ ID NO:22.

78. A polynucleotide of claim 12, comprising the polynucleotide sequence of SEQ ID NO:23.

79. A polynucleotide of claim 12, comprising the polynucleotide sequence of SEQ ID NO:24.

80. A polynucleotide of claim 12, comprising the polynucleotide sequence of SEQ ID NO:25.

81. A polynucleotide of claim 12, comprising the polynucleotide sequence of SEQ ID NO:26.

82. A polynucleotide of claim 12, comprising the polynucleotide sequence of SEQ ID NO:27.

83. A polynucleotide of claim 12, comprising the polynucleotide sequence of SEQ ID NO:28.

84. A polynucleotide of claim 12, comprising the polynucleotide sequence of SEQ ID NO:29.

85. A polynucleotide of claim 12, comprising the polynucleotide sequence of SEQ ID NO:30.

86. A polynucleotide of claim 12, comprising the polynucleotide sequence of SEQ ID NO:31.

87. A polynucleotide of claim 12, comprising the polynucleotide sequence of SEQ ID NO:32.

88. A polynucleotide of claim 12, comprising the polynucleotide sequence of SEQ ID NO:33.

89. A polynucleotide of claim 12, comprising the polynucleotide sequence of SEQ ID NO:34.

90. A polynucleotide of claim 12, comprising the polynucleotide sequence of SEQ ID NO:35.

91. A polynucleotide of claim 12, comprising the polynucleotide sequence of SEQ ID NO:36.
